(19) United States
(12) Patent Application Publication
(10) Pub. No.: US 2019/0281797 A1
(43) Pub. Date: Sep. 19, 2019

(54) PSORIASIS-INDUCED ANIMAL MODEL AND USE THEREOF

(71) Applicants: RESEARCH & BUSINESS FOUNDATION SUNGKYUNKWAN UNIVERSITY, Suwon-si (KR); THE ASAN FOUNDATION, Seoul (KR)

(72) Inventors: Chang Woo LEE, Pohang-si (KR); Heoun Jeong GO, Seoul (KR); Su Hyeon KIM, Busan (KR); Seo Yoon BAE, Ulsan (KR)

(73) Assignees: RESEARCH & BUSINESS FOUNDATION SUNGKYUNKWAN UNIVERSITY, Suwon-si (KR); THE ASAN FOUNDATION, Seoul (KR)

(21) Appl. No.: 16/315,687

(22) PCT Filed: Jun. 30, 2017
(86) PCT No.: PCT/KR2017/006962

§ 371 Date: Jan. 7, 2019

(30) Foreign Application Priority Data
Jul. 5, 2016 (KR) 10-2016-0084594
Jun. 29, 2017 (KR) 10-2017-0082620

(51) Int. Cl. A01K 67/027; C12N 15/85; A61K 31/7105; A61P 17/06

(52) U.S. Cl. CPC A01K 67/0275; C12N 15/8509; A61K 31/7105; A01K 2227/105; A01K 2217/206; A01K 2267/0306; A61P 17/06

(57) ABSTRACT

Provided are a psoriasis-induced transgenic animal model overexpressing the Pellino homolog 1 (Peli1) gene according to doxycycline administration, and a use thereof. The transgenic animal model of the present disclosure exhibited similarity to phenotypes shown in patients with psoriasis, due to overexpression of the Pellino homolog 1 (Peli1) gene according to doxycycline administration. It is anticipated that the transgenic animal model may be usefully used in clinical studies, such as screening for a candidate drug for the treatment of psoriasis. Additionally, it is anticipated that a peptide derived from the Peli1 FHA domain targeting the FHA binding motif that inhibits normal substrate binding between a substrate protein and the Peli1 protein may be usefully used in the development of new drugs for psoriasis-associated diseases. Moreover, by confirming an expression level of the Peli1 protein, it is anticipated to be usefully used in evaluating the severity of patients with psoriasis.

### TECHNICAL FIELD

The present disclosure relates to a psoriasis-induced animal model and a use thereof, and more particularly, to a psoriasis-induced transgenic animal model overexpressing the Pellino homolog 1 (Peli1) gene according to administration of doxycycline, and a use thereof.

### BACKGROUND ART

Psoriasis is a representative chronic skin disease and, in most cases, persists for 10 to 20 years after the onset, and, even if it improves temporarily due to treatment, patients with psoriasis have to live with the possibility of recurrence during their lifetime. Psoriasis is characterized by erythematous skin lesions covered with white-silvery scales, the boundary of which is clear, and mainly occurs on the elbows, knees, buttocks, scalp, and the like, which are very stimulated areas. In addition, psoriasis may be accompanied by various complications. Among them, there is psoriasis arthritis, which is a unique arthritis occurring in patients with psoriasis. There is no way to directly prevent the onset of such psoriasis, and the best method for treatment is to prevent the aggravation thereof. According to various statistics, the number of patients diagnosed with psoriasis is globally about 3%, and about 1% to about 2% in Korea, and of these, about one third of patients are severe psoriasis patients who are rarely curable. In particular, according to data from the National Health Insurance Review and Assessment Service, in 2013, 163,936 Korea people are found to have received treatment for psoriasis.

Although the cause of psoriasis has not yet been accurately verified, the activity of T cells, which are immune cells in the skin, is increased, and consequently, cytokines secreted by T cells stimulate epidermal cells, thus causing the excessive proliferation and inflammation of epidermal cells, and thus immunological abnormalities and gene mutations have recently emerged as major research fields. In addition, environmental factors, drugs, persistent skin irritation, skin dryness, upper respiratory inflammation, mental stress, and the like are proven to cause or exacerbate psoriasis.

Various methods have been used for the treatment of psoriasis, and various methods have been attempted to find more effective therapies. These methods are divided into topical treatment wherein a medicine is directly applied onto the skin, phototherapy using rays, and systemic treatment wherein a drug such as an immunosuppressive drug or a steroid drug is taken, and these therapies are used in combination in accordance with conditions of patients. Treatment methods are determined according to the severity of psoriasis, activity, the type and condition of a lesion, and the site of the disease, and the age of a patient, treatment accessibility, and mental conditions are also very important. In mild cases, treatment usually begins with an applicable medicine, and in severe cases, phototherapy or edible drugs are used together. In addition, in recent years, biological agents developed through various studies have been tested for stability through clinical testing and are actually used in patients. Existing therapies mostly have an effect of alleviating symptoms, but are not fundamental therapies, and there has been no report of, particularly, psoriasis-patient-customized or psoriasis-patient-targeted therapies.

Therefore, there is a need for effective diagnosis and treatment of psoriasis, and it is essential to produce animal models suitable for the development of biological samples to study the mechanisms and therapies of psoriasis, and thus studies on these have actively been conducted (see KR 10-2010-0021561), but are still insufficient.

### DISCLOSURE

### Technical Problem

The present disclosure has been made to address the above-described problems, and the inventors of the present disclosure confirmed that transgenic mice overexpressing the Peli1 gene via administration of doxycycline could be used as an animal model for the treatment of severe psoriasis, and also confirmed the possibility of causing psoriasis due to overexpression of the Peli1 protein, and thus it was confirmed that a Peli1 protein expression or activity inhibitor could prevent, alleviate, inhibit, or treat psoriasis, and a peptide derived from the Peli1 FHA domain targeting for the FHA binding motif of a substrate protein could be used as a novel therapeutic agent by interferring with normal substrate binding of the Peli1 protein, thus completing the present disclosure based on these findings.

Therefore, an object of the present disclosure is to provide a psoriasis-induced transgenic animal model overexpressing the Pellino homolog 1 (Peli1) gene, and a method of producing the psoriasis-induced animal model.

In addition, another object of the present disclosure is to provide a method of screening for a psoriasis therapeutic agent by using the animal model.

In addition, psoriasis induced by overexpression of the Peli1 protein due to an immunosuppressant or a steroid agent, which is the most frequently prescribed systemic psoriasis therapeutic agent, may be efficiently treated, and thus the present disclosure provides a method of using Peli1 for a treatment target of the development of a psoriasis therapeutic agent.

In addition, still another object of the present disclosure is provide a peptide having any one amino acid sequence selected from the group consisting of SEQ ID NOS: 5 to 11, and a pharmaceutical composition for preventing or treating psoriasis, which includes the peptide as an active ingredient.

In addition, yet another object of the present disclosure provides a peptide inhibiting the binding activity of Peli1, characterized in that a cell-penetrating peptide is linked to a peptide having any one amino acid sequence selected from the group consisting of SEQ ID NOS: 5 to 11, and a pharmaceutical composition for preventing or treating psoriasis, which includes, as an active ingredient, the peptide inhibiting the binding activity of Peli1.

In addition, yet another object of the present disclosure is to provide a composition for diagnosing psoriasis, which includes an agent for measuring an expression level of the Peli1 protein.

However, technical problems to be solved by the present disclosure are not limited to the above-described technical problems, and other unmentioned technical problems will become apparent from the following description to those of ordinary skill in the art.

### Technical Solution

To achieve the above-described objects, the present disclosure provides a psoriasis-induced transgenic animal model overexpressing the Pellino homolog 1 (Peli1) gene.

In one embodiment of the present disclosure, the Peli1 gene may be overexpressed according to administration of doxycycline.

In another embodiment of the present disclosure, the Peli1 gene may have a base sequence of SEQ ID NO: 1.

The present disclosure also provides a method of producing a psoriasis-induced animal model, including the following processes:

(a) transfecting an embryo of a host animal with an expression vector including the Pellino homolog 1 (Peli1) gene;

(b) implanting the transfected embryo into the uterus of a surrogate mother and allowing the surrogate mother to give birth to the embryo as a first generation progeny animal; and

(c) crossing the first generation progeny animal with a reverse tetracycline transactivator (rtTA) animal model to obtain a second generation progeny animal.

The present disclosure also provides a method of screening for a psoriasis therapeutic agent, including the following processes:

a) treating the animal model with a candidate material; and

b) identifying a prognosis while rearing the animal model treated with the candidate material.

The present disclosure also provides a pharmaceutical composition for preventing or treating psoriasis, which includes a Pellino homolog 1 (Peli1) protein expression or activity inhibitor as an active ingredient.

In one embodiment of the present disclosure, the Peli1 protein may be encoded by a polynucleotide consisting of a base sequence of SEQ ID NO: 1.

In another embodiment of the present disclosure, the Peli1 protein may have an amino acid sequence of SEQ ID NO: 2.

In another embodiment of the present disclosure, the Peli1 protein expression inhibitor may be any one selected from the group consisting of an antisense nucleotide, a short interfering RNA, a short hairpin RNA, and a ribozyme, which complementarily bind to mRNA of the Peli1 gene.

In another embodiment of the present disclosure, the Peli1 protein activity inhibitor may be any one selected from the group consisting of a compound, a peptide, peptide mimetics, a substrate analogue, an aptamer, and an antibody, which complimentarily bind to the Peli1 protein.

The present disclosure also provides a peptide having any one amino acid sequence selected from the group consisting of SEQ ID NOS: 5 to 11.

In one embodiment of the present disclosure, the peptide may inhibit binding between the Peli1 protein and a substrate protein.

The present disclosure also provides a peptide inhibiting the binding activity of Peli1, characterized in that a cell-penetrating peptide is linked to a peptide having any one amino acid sequence selected from the group consisting of SEQ ID NOS: 5 to 11.

In another embodiment of the present disclosure, the peptide inhibiting the binding activity of Peli1 may be linked via a GS linker.

In another embodiment of the present disclosure, the cell-penetrating peptide may be any one selected from the group consisting of polyarginines, Tat₄₉₋₅₇, penetratin, Pep-1, transportan, nuclear localization sequence (NLS), and HP4.

The present disclosure also provides a pharmaceutical composition for preventing or treating psoriasis, which includes, as an active ingredient, the above-described peptide or an inhibitor peptide against the binding activity of Peli1.

The present disclosure also provides a method of treating psoriasis, including administering the pharmaceutical composition to an individual.

The present disclosure also provides a use of the pharmaceutical composition for preventing or treating psoriasis.

The present disclosure also provides a novel use of a gene encoding the Pellino homolog 1 (Peli1) protein for preparing a composition for the treatment of psoriasis.

The present disclosure also provides a composition for diagnosing psoriasis, which includes an agent for measuring an expression level of the Peli1 protein.

The present disclosure also provides a method of detecting the Peli1 protein from an individual-derived sample, to provide information needed for the diagnosis of psoriasis.

The present disclosure also provides a use of the Peli1 protein for diagnosing psoriasis.

### Advantageous Effects

It was confirmed that a transgenic animal model according to the present disclosure overexpressed the Pellino homolog 1 (Peli1) gene according to administration of doxycycline, and thus exhibited similarity to phenotypes exhibited in patients with psoriasis, and thus animal models for the development of biological samples can be designed to study effective therapies for psoriasis, and are expected to be usefully used in clinical studies such as screening for a candidate drug for the treatment of psoriasis, and the like.

In addition, the possibility of preventing, alleviating, inhibiting, or treating psoriasis by inhibiting the expression or activity of the Peli1 protein was specifically confirmed, and it is anticipated that peptides derived from the Peli1 FHA domain targeting the FHA binding motif, which interfere with normal substrate binding between a substrate protein and the Peli1 protein, can be usefully used in developing new drugs for psoriasis-associated diseases.

Moreover, an expression level of the Peli1 protein was confirmed, and thus it is anticipated that the Peli1 protein can be usefully used in evaluating the severity of patients with psoriasis.

### DESCRIPTION OF DRAWINGS

FIG. 1A is a schematic view illustrating a process of producing doxycycline-inducible Peli1 transgenic mice in which Peli1 is overexpressed according to administration of doxycycline.

FIG. 1B illustrates results of confirming whether transformation occurred such that Peli1 was overexpressed according to administration of doxycycline, through PCR.

FIG. 2 illustrates results of confirming Peli1 protein expression in respective tissues of doxycycline inducible Peli1 transgenic mice, through western blotting.

FIG. 3A illustrates results of visually comparing spontaneous lesion induction according to administration of doxycycline in doxycycline-inducible Peli1 transgenic mice (rtTA-Peli1) and control mice (rtTA).

FIG. 3B illustrates results of calculating the proportion of mice showing the phenotypes illustrated in FIG. 3A.

FIG. 3C illustrates results of scoring the levels of lesion according to administration of doxycycline in doxycycline-inducible Peli1 transgenic mice (rtTA-Peli1) and control mice (rtTA).

FIG. 4A illustrates results of comparing the structures of skin layers with each other through hematoxylin and eosin (H&E) staining to verify whether psoriasis was induced via administration of doxycycline in doxycycline-inducible Peli1 transgenic mice (rtTA-Peli1) and control mice (rtTA).

FIG. 4B illustrates results of performing immunostaining using a Peli1 antibody and an antibody against Psoriasin, which is a psoriasis marker protein, to confirm whether the overexpression of Peli1 was induced according to administration of doxycycline in doxycycline-inducible Peli1 transgenic mice (rtTA-Peli1) and control mice (rtTA).

FIG. 4C illustrates results of comparing changes in the development of skin layers through immunostaining using antibodies against K14, which is a marker of the basal layer, K10, which is a marker of the spinous layer, and Loricrin, which is a marker of the granular layer, to verify whether psoriasis was induced according to administration of doxycycline in doxycycline-inducible Peli1 transgenic mice (rtTA-Peli1) and control mice (rtTA).

FIG. 4D illustrates results of comparing the abilities of basal layer cells to proliferate through immunostaining using antibodies against Ki67 and K14, to verify whether psoriasis was induced according to administration of doxycycline in doxycycline-inducible Peli1 transgenic mice (rtTA-Peli1) and control mice (rtTA).

FIG. 4E illustrates results of comparing changes in angiogenesis through immunostaining using a CD31 antibody, to verify whether psoriasis was induced according to administration of doxycycline in doxycycline-inducible Peli1 transgenic mice (rtTA-Peli1) and control mice (rtTA).

FIG. 4F illustrates results of comparing changes in infiltration of T cells through immunostaining using a CD3 antibody, to verify whether psoriasis was induced according to administration of doxycycline in doxycycline-inducible Peli1 transgenic mice (rtTA-Peli1) and control mice (rtTA).

FIGS. 4G and 4H illustrate results of comparing changes in lymph nodes around skin lesions, to verify whether psoriasis was induced according to administration of doxycycline in doxycycline-inducible Peli1 transgenic mice (rtTA-Peli1) and control mice (rtTA).

FIG. 4I illustrates results of comparing the activation of immune cells in lymph nodes through flow cytometry, to verify whether psoriasis was induced according to administration of doxycycline in doxycycline-inducible Peli1 transgenic mice (rtTA-Peli1) and control mice (rtTA).

FIGS. 4J and 4K are graphs showing the results of FIG. 4I.

FIG. 4L illustrates results of comparing the proportions of activated T cells in the results of FIG. 4I according to administration of doxycycline.

FIG. 5A illustrates results of comparing the levels of cytokines such as IFNγ, IL-4, IL-22, and IL-23 by using cDNA of CD3⁺ T cells of the lymph nodes through quantitative reverse transcription-polymerase chain reaction, to verify whether psoriasis was induced according to administration of doxycycline in doxycycline-inducible Peli1 transgenic mice (rtTA-Peli1) and control mice (rtTA).

FIG. 5B illustrates results of performing immunostaining using an antibody against IL-17, to verify whether psoriasis was induced according to administration of doxycycline in doxycycline-inducible Peli1 transgenic mice (rtTA-Peli1) and control mice (rtTA).

FIG. 5C illustrates results of performing immunostaining using an antibody against IL-23, to verify whether psoriasis was induced according to administration of doxycycline in doxycycline-inducible Peli1 transgenic mice (rtTA-Peli1) and control mice (rtTA).

FIG. 6A illustrates results of visually comparing spontaneous lesion induction according to the administration and withdrawal of doxycycline in doxycycline-inducible Peli1 transgenic mice (rtTA-Peli1) and control mice (rtTA).

FIG. 6B illustrates results of scoring lesion levels according to the administration and withdrawal of doxycycline in doxycycline-inducible Peli1 transgenic mice (rtTA-Peli1) and control mice (rtTA).

FIG. 6C illustrates results of comparing the structures of skin layers through H&E staining, to verify whether psoriasis was induced according to the administration and withdrawal of doxycycline in doxycycline-inducible Peli1 transgenic mice (rtTA-Peli1) and control mice (rtTA).

FIG. 7A is a view illustrating a case in which an imiquimod-inducible psoriasis model was applied to normal mice (WT) and Peli1 knockdown (Peli1 KO) mice, to confirm whether psoriasis induction was inhibited when Peli1 protein expression was inhibited.

FIG. 7B illustrates results of visually comparing psoriasis lesions induced according to treatment with imiquimod (toll-like receptor 7/9 agonist) in normal mice (WT) and Peli1 knockdown (Peli1 KO) mice.

FIG. 7C illustrates results of scoring psoriasis lesion levels according to imiquimod treatment in normal mice (WT) and Peli1 knockdown (Peli1 KO) mice.

FIG. 7D is a graph showing results of measuring the thickness of the skin according to imiquimod treatment in normal mice (WT) and Peli1 knockdown (Peli1 KO) mice.

FIG. 7E illustrates results of comparing the structures of skin layers through H&E staining, to verify the levels of psoriasis lesions after treatment with imiquimod four times in normal mice (WT) and Peli1 knockdown (Peli1 KO) mice.

FIG. 8A is a diagram for a case in which doxycycline-inducible Peli1 transgenic mice (rtTA-Peli1) and control mice (rtTA) were administered doxycycline for 12 weeks, and then intraperitoneally injected with cyclosporine (CsA) or methotrexate (MTX), which is an existing psoriasis systemic therapeutic agent, to verify a use of the doxycycline-inducible Peli1 transgenic mice (rtTA-Peli1) as a preclinical animal model for the evaluation of therapeutic efficacy.

FIG. 8B illustrates results of comparing the structures of skin layers through H&E staining, to compare the levels of psoriasis lesions after doxycycline-inducible Peli1 transgenic mice (rtTA-Peli1) and control mice (rtTA) were administered doxycycline for 12 weeks, and then intraperitoneally injected with cyclosporine (CsA) or methotrexate (MTX), which are immunosuppressants, for a certain period of time.

FIG. 8C illustrates results of scoring the levels of psoriasis lesions after doxycycline-inducible Peli1 transgenic mice (rtTA-Peli1) and control mice (rtTA) were administered doxycycline for 12 weeks, and then intraperitoneally injected with cyclosporine (CsA) or methotrexate (MTX), which are immunosuppressants, for a certain period of time.

FIG. 8D is a graph showing results of measuring the thickness of the skin after doxycycline-inducible Peli1 transgenic mice (rtTA-Peli1) and control mice (rtTA) were administered doxycycline for 12 weeks, and then intraperitoneally injected with cyclosporine (CsA) or methotrexate (MTX), which are immunosuppressants, for a certain period of time.

FIG. 9A is a schematic view illustrating a process of identifying a target mRNA sequence and producing shRNA against the mRNA sequence to inhibit the synthesis of a protein in Peli1 mRNA.

FIG. 9B illustrates results of confirming whether the expression of the Peli1 protein was inhibited in cells transfected with the shRNA produced in FIG. 9A, through western blotting.

FIG. 10 schematically illustrates a process of producing a peptide that inhibits the binding activity of Peli1 and a process in which the peptide inhibits the binding activity of Peli1.

FIG. 11A schematically illustrates a process of producing a peptide that inhibits the binding activity of Peli1.

FIG. 11B illustrates results of confirming the cell penetration of a peptide that inhibits the binding activity of Peli1 to which a cell-penetrating peptide for enhancing cell penetration was bound, through fluorescence staining.

FIG. 11C illustrates results of comparing toll-like receptor (TLR) signaling activation after treatment with an inhibitory peptide against the binding activity of Peli1, through western blotting.

### MODES OF THE INVENTION

As a result of having conducted studies to develop animal models suitable for use in studying and developing a therapeutic agent of psoriasis, the inventors of the present disclosure verified that transgenic mice overexpressing the Peli1 gene according to doxycycline administration exhibited similarity to phenotypes of patients with psoriasis, and thus completed the present disclosure based on these findings.

Hereinafter, the present disclosure will be described in detail.

The present disclosure provides a psoriasis-induced transgenic animal model overexpressing the Pellino homolog 1 (Peli1) gene according to doxycycline administration.

The present disclosure also provides a method of producing a psoriasis-induced animal model, including the following processes:

(a) transfecting an embryo of a host animal with an expression vector including the Pellino homolog 1 (Peli1) gene;

(b) implanting the transfected embryo into the uterus of a surrogate mother and allowing the surrogate mother to give birth to the embryo as a first generation progeny animal; and

(c) crossing the first generation progeny animal with a reverse tetracycline transactivator (rtTA) animal model to obtain a second generation progeny animal.

The term “Pellino homolog 1 (Peli1) gene” as used herein refers to a gene encoding the Pellino homolog 1 protein, which is known as an E3 ubiqutin-conjugating enzyme, and the Peli1 gene is located in chromosome 2 in humans and in chromosome 11 in mice. Specific base sequence and protein information of the Peli1 protein or gene are known in NCBI (NCBI Reference Sequence: NP_065702, NM_020651). In the present disclosure, a polynucleotide of SEQ ID NO: 1 is used as the Peli1 gene.

In the present disclosure, the animal may be, but is not limited to, a mouse, a rat, a cow, a horse, a pig, a monkey, a duck, a dog, a cat, or the like, and is preferably a mouse.

In one embodiment of the present disclosure, as a result of producing a transgenic animal model overexpressing the Peli1 gene according to doxycycline administration (see Example 1), and examining whether lesions were spontaneously induced in the animal model according to doxycycline administration, it was confirmed that, when doxycycline was continuously administered, lesions spontaneously occurred, as compared to a control. In particular, it was confirmed that hair gloss disappeared, hair loss progressed, and abnormal findings of the skin were observed (see Example 4).

In addition, in another embodiment of the present disclosure, as a result of examining the induction of psoriasis in the produced animal model, it was confirmed that, when doxycycline was continuously administered, the epidermal layer thickened, angiogenesis of the epidermal layer was increased, the infiltration of T cells into the skin layer was increased, and immune cells were abnormally activated and underwent a helper T cell response in lymph nodes around skin lesions, as compared to a control, and thus it was confirmed that these were very similar to the phenotype of psoriasis (see Example 5). It was confirmed that the phenotype of psoriasis disappeared when the expression of Peli1 was reduced to a normal level by stopping doxycycline administration and when normal Peli1 expression was inhibited (see Example 6). In addition, it was confirmed that, when cyclosporine (CsA) or methotrexate (MTX), which are immunosuppressants used as an existing psoriasis systemic therapeutic agent, was periodically administered intraperitoneally, the phenotype of psoriasis was alleviated, and thus the produced animal model was verified as a preclinical animal model (see Example 7).

Thus, the animal model according to the present disclosure may be usefully used in treating and studying psoriasis.

Therefore, according to another embodiment of the present disclosure, there is provided a method of screening for a psoriasis therapeutic agent by using the animal model produced using the above-described method.

The screening method may include: a) treating the animal model with a candidate material; and b) identifying a prognosis while rearing the animal model treated with the candidate material, and the candidate material may be, but is not limited to, any one selected from the group consisting of a natural compound, a synthetic compound, RNA, DNA, a polypeptide, an enzyme, a protein, a ligand, an antibody, an antigen, a metabolite of a bacterium or a mycete, and a bioactive molecule.

In addition, the possibility of psoriasis induction due to overexpression of the Peli1 protein was confirmed through the production of a psoriasis-induced animal model, and thus a Peli1 protein expression or activity inhibitor may be used as an active ingredient of a composition effective in preventing, alleviating, inhibiting, or treating psoriasis.

Therefore, according to another embodiment of the present disclosure, there are provided a pharmaceutical composition for preventing or treating psoriasis, which includes, as an active ingredient, a Pellino homolog 1 (Peli1) protein expression or activity inhibitor, and a method of treating psoriasis by using the pharmaceutical composition.

In the present disclosure, the Peli1 protein may have an amino acid sequence of SEQ ID NO: 2.

The term “prevention” as used herein means all actions that inhibit psoriasis or delay the onset thereof via administration of the pharmaceutical composition according to the present disclosure.

The term “treatment” as used herein means all actions that alleviate or beneficially change symptoms due to psoriasis via administration of the pharmaceutical composition according to the present disclosure.

In the present disclosure, the Peli1 protein expression inhibitor may be selected from the group consisting of an antisense nucleotide, a siRNA, a shRNA, and a ribozyme, which complementarily bind to mRNA of the Peli1 gene, but the present disclosure is not limited thereto.

In the present disclosure, the Peli1 protein activity inhibitor may be, but is not limited to, any one selected from the group consisting of a compound, a peptide, peptide mimetics, a substrate analogue, an aptamer, and an antibody, which complimentarily bind to the Peli1 protein, and may be any drug that inhibits the activity of the Peli1 protein.

In the present disclosure, the peptide mimetics inhibit the activity of the Peli1 protein by inhibiting the binding domain of the Peli1 protein, and the peptide mimetics may be peptides or non-peptides, and may also include amino acids linked by non-peptide bonds such as psi bonds.

The term “aptamer” as used herein refers to a single-stranded DNA or RNA molecule, and the aptamer may be obtained by isolating oligomers that bind to specific chemical molecules or biological molecules with high affinity and specificity by an evolutionary method using an oligonucleotide library called systematic evolution of ligands by exponential enrichment (SELEX). The aptamer may specifically bind to a target to regulate the activity of the target, and may inhibit the function of the target by, for example, binding.

In the present disclosure, the antibody may specifically and directly bind to the Peli1 protein, thereby effectively inhibiting the activity of the Peli1 protein. The antibody specifically binding to the Peli1 protein may be a polyclonal antibody or a monoclonal antibody. The antibody specifically binding to the Peli1 protein may be prepared using a method known to those of ordinary skill in the art, or a commercially available Peli1 protein antibody may be purchased and used.

In addition, the Peli1 protein has a RING-like domain, and thus acts as an E3 ubiquitin-conjugating enzyme. That is, the Peli1 protein is activated through various posttranslational modifications of a protein, and thus acts as an E3 ubiquitin-conjugating enzyme, and binds to a target protein through the FHA domain Therefore, a peptide derived from the Peli1 FHA domain targeting the FHA binding motif of a substrate protein inhibits normal substrate binding of the Peli1 protein, and thus may be used as a novel therapeutic agent.

In another embodiment of the present disclosure, it was confirmed that the Peli1 protein bound to a target protein through the FHA domain, and thus an FHA domain-derived peptide of the Peli1 protein was produced to inhibit the binding between the Peli1 protein and the target protein (see Example 9).

Therefore, according to another embodiment of the present disclosure, there are provided a peptide having any one amino acid sequence selected from the group consisting of SEQ ID NOS: 5 to 11, and a pharmaceutical composition for preventing or treating psoriasis, which includes the above-described peptide.

In the present disclosure, the peptide may inhibit the binding between the Peli1 protein and a substrate protein by targeting the FHA binding motif. In addition, the peptide may be linked to a cell-penetrating peptide for facilitating intracellular penetration.

In this regard, the cell-penetrating peptide may be any one selected from the group consisting of polyarginines, Tat₄₉₋₅₇, penetratin, Pep-1, transportan, nuclear localization sequence, and HP4, and preferably is HP4, but the present disclosure is not limited thereto.

Hereinafter, exemplary embodiments will be described with reference to the following examples to aid in understanding of the present disclosure. However, these examples are provided only to more easily understand the present disclosure and are not intended to limit the scope of the present disclosure.

### EXAMPLES

### Example 1. Experimental Preparation and Experimental Methods

1-1. Animal Experiment

All animal experiments were conducted in accordance with protocols approved by the Institutional Animal Care and Use Committee (IACUC) at Sungkyunkwan University School of Medicine (SUSM). Sungkyunkwan University School of Medicine (SUSM) conducted all animal experiments in accordance with guidelines of the Association for Assessment and Accreditation of Laboratory Animal Care International (AAALAC International) and the Institute of Laboratory Animal Resources (ILAR).

1-2. Genotyping

Genomic DNA (gDNA) was extracted from the tails of doxycycline inducible Peli1 transgenic mice, and then a polymerase chain reaction (PCR) was performed to confirm transformation.

In the case of TetO & Peli1, RT-PCR was performed using gDNA as a template and a pair of primers a total of 4 times at 95° C. for 30 seconds, 55° C. for 30 seconds, and 72° C. for 1 minute, and then a total of 34 times at 95° C. for 30 seconds, 58° C. for 1 minute, and 72° C. for 1 minute. In addition, in the case of R26 and rtTA, PCR was performed a total of 34 times at 95° C. for 30 seconds, 65° C. for 1 minute, and 72° C. for 1 minute, and specific sequences of the used primers are as follows:

1) TetO & Peli1 primer sequences:
(SEQ ID NO: 12)
Forward primer (cmv TetO-1) 5′-AAG TGA AAG TCG AGC
TCG-3′,
(SEQ ID NO: 13)
reverse primer (Peli1 1/3 length) 5′-TGA TAT CGT
GTG CTG GTC TTT G -3′
2) R26 & rtTA primer sequences:
(1) Normal mice (WT) - R26(F) and R26 (R);
(SEQ ID NO: 14)
forward primer 5′- AAA GTC GCT CTG AGT TGT TAT -
3′,
(SEQ ID NO: 15)
reverse primer 5′- GGA GCG GGA GAA ATG GAT ATG -3′
(2) Transgenic mice (Tg) - R26(F) and rtTA (R);
(SEQ ID NO: 16)
forward primer 5′- AAA GTC GCT CTG AGT TGT TAT -
3′,
(SEQ ID NO: 17)
reverse primer 5′- GCG AAG AGT TTG TCC TCA ACC -3′

10 μl of the PCR reaction product was subjected to electrophoresis on 1% agarose gel in the presence of etidium bromide to observe bands.

1-3. Western Blotting

To confirm the expression of the Peli1 protein in mice expressing Myc-Peli1 according to the administration of doxycycline, control (−/rtTA) mice and experimental (Myc-Peli1/rtTA) mice were administered doxycycline (2 mg/ml) for 2 weeks. A lysis buffer (150 mM NaCl, 20 mM HEPES, 5 mM EDTA, 0.5% Nonidet P-40, 1 mM phenylmethanesulfonyl fluoride, 10 mM NaF, 1 mM Na₃Vo₄, 1 mM dithiothreitol, and protease inhibitor cocktail) was added to respective organ tissues, followed by homogenization using a homogenizer and isolation using a ultracentrifuge (14,000 rpm, 30 min, 4° C.). An extract of the supernatant was obtained, and then proteins were quantified using a Bradford quantification method. 50 μg of each organ extract was subjected to 8% SDS-PAGE electrophoresis, followed by transfer (using a nitrocellulose membrane) and blocking with a TBS-T buffer containing 5% skim milk (100 Mm NaCl, 20 Mm Tris-HCl pH 7.4, 0.05% Tween-20), reaction with an antibody, i.e., anti-Myc (Roche; 1:2,000 dilution), anti-Peli1 (Santa Cruz; 1:2,000 dilution), or anti-β-actin (Sigma; 1:4,000 dilution) in 3% BSA (containing a TBS-T solution) at 4° C. for 12 hours or more, washing three times with a TBS-T buffer (Washing; 3 times, 10 min, RT), reaction with a solution obtained by diluting an HRP-conjugated secondary antibody in a blocking solution to 1:3,000, at room temperature for 2 hours, and washing three times with TBS-T (Washing; 3 times, 10 min, RT), and then luminescence was induced using an ECL solution and sensitized to an X-ray film.

1-4. Flow Cytometry

Cells were obtained from the lymph nodes of mice, erythrocytes were removed therefrom, and the resulting cells were stained using the following antibodies. More particularly, the cells were stained with anti-CD3 Percp cy 5.5, anti-CD4 PE cy7, anti-CD8 PE, anti-CD122 APC, anti-CD44 FITC, and anti-CD62L APC cy7, and then analyzed using Canto II Flow cytometry (BD Bioscience), which is flow cytometry equipment, through fluorescence of the antibodies.

1-5. Histopathology

Tissues of mice were fixed using 10% neutral buffered formalin to produce paraffin blocks. Sections having a thickness of 3 μm were obtained from the paraffin blocks, and then stained with hematoxylin & eosin (H&E). In the case of immunohistochemistry, staining was performed using an anti-K14 antibody, an anti-Ki67 antibody, an anti-CD31 antibody, and an anti-CD3 antibody, followed by mounting using a mounting medium containing a DAPI reagent. Images of the stained tissue samples were acquired using an AxioCam digital microscope camera and AxioVision image processing software (Carl Zeiss).

### Example 2. Production of Inducible Peli1 Transgenic Mice

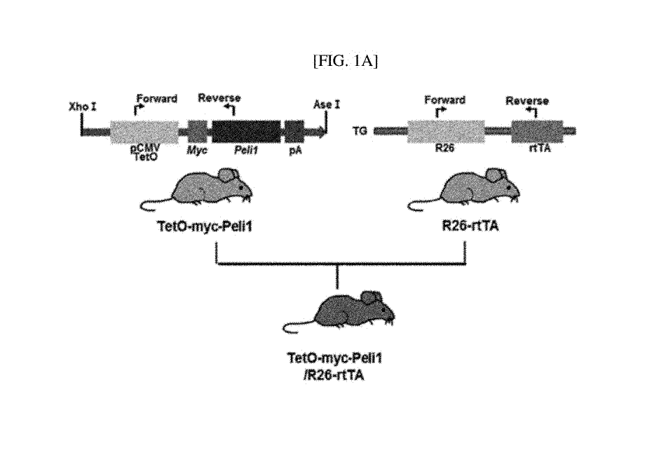

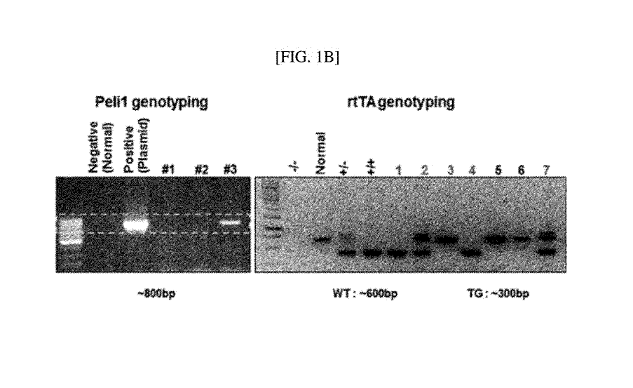

A transgenic animal model overexpressing the Pellino homolog 1 (Peli1) gene according to the administration of doxycycline was produced as follows, and a schematic view of the production method is illustrated in FIG. 1A.

More particularly, transgenic mice were produced by microinjecting a vector (see FIG. 1A) cloned with the Myc epitope-tagged human Peli1 gene into 1-cell embryos. After the microinjection, tetracycline-responsive element (TetO)-Myc-Peli1 transgenic mice were selected through the genotyping method described in Example 1-2. The selected mice were crossed with reverse tetracycline transactivator (R26-rtTA) mice to obtain progeny transgenic mice (doxycycline inducible TetO-Myc-Peli1 transgenic mice), and it was examined whether transformation occurred, through the polymerase chain reaction (PCR) described in Example 1-2.

As a result, as illustrated in FIG. 1B, it was confirmed that transgenic mice expressing both Myc-Peli1 and rtTA were obtained.

### Example 3. Verification of Expression Level of Peli1 in Peli1 Inducible Transgenic Mice

The expression of Myc-Peli1 at the protein level was confirmed in tissues of doxycycline-inducible Peli1 transgenic mice overexpressing Peli1 according to the administration of doxycycline, which were produced in Example 2. To this end, control (−/rtTA) mice and experimental (Myc-Peli1/rtTA) mice were administered doxycycline (2 mg/ml) for 2 weeks to obtain various tissues. More particularly, through the western blotting described in Example 1-3, the expression of Myc-Peli1 was confirmed in the brain, lungs, heart, thymus, stomach, small intestine, epididymis, colon, kidneys, skin, testis, prostate, pancreas, liver, spleen, lymph nodes, and bone marrow of the control (−/rtTA) mice and the experimental (Myc-Peli1/rtTA) mice.

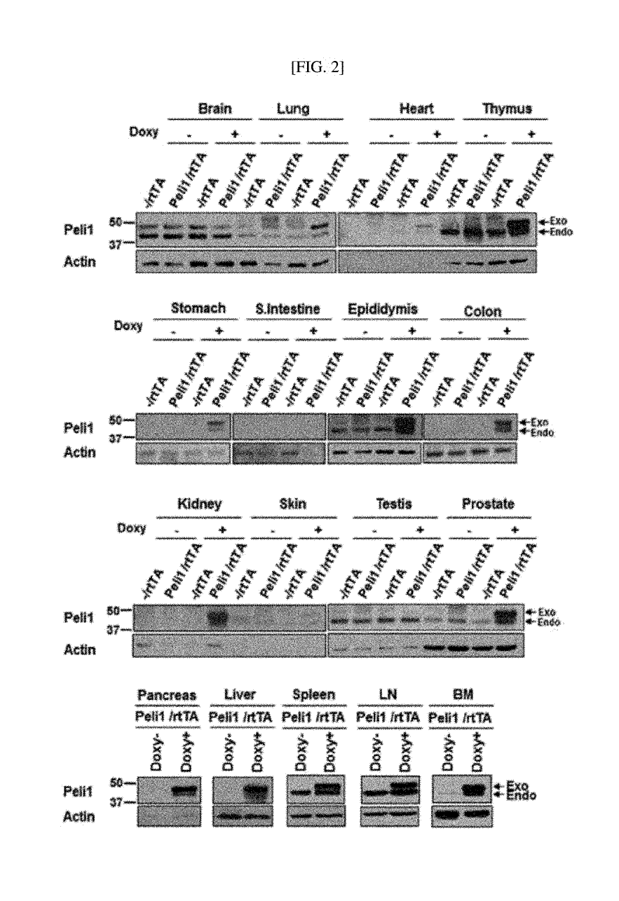

As a result, as illustrated in FIG. 2, it was confirmed that Myc-Peli1 was highly expressed in the lung, the thymus, the stomach, the epididymis, the colon, the kidneys, the prostate, the pancreas, the liver, the spleen, lymph nodes, bone marrow, and the like. In contrast, it was confirmed that Myc-Peli1 was expressed at a low level or hardly expressed in the brain, the heart, the small intestine, the skin, the testis, and the like.

### Example 4. Verification of Spontaneous Lesion Induction According to Doxycycline Administration in Peli1 Inducible Transgenic Mice

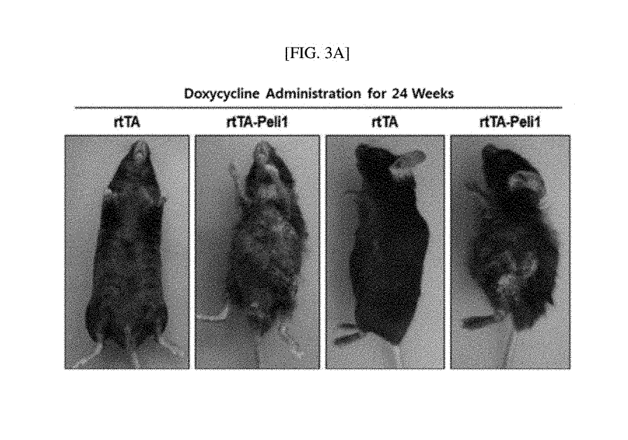

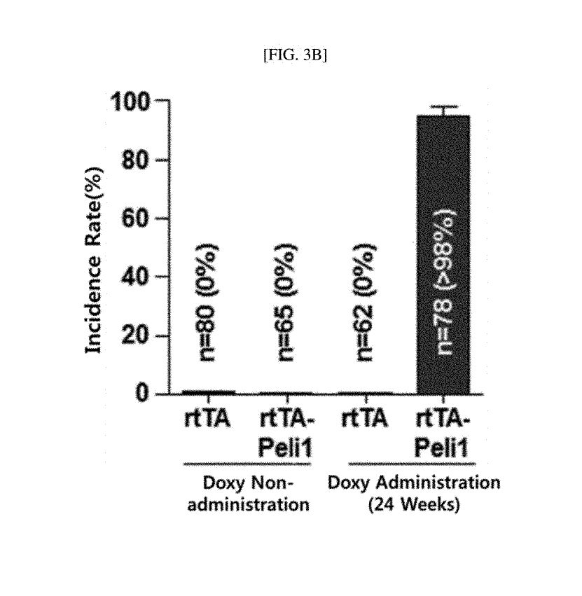

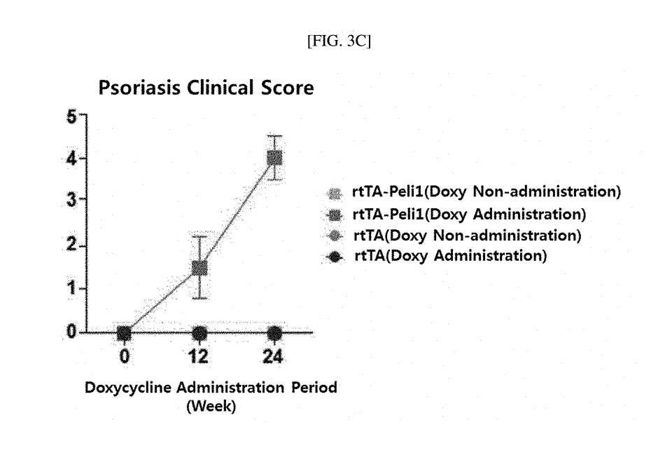

First, control (rtTA) mice and experimental (rtTA-Peli1) mice were administered drinking water containing doxycycline (2 mg/ml) for 24 weeks, and then the occurrence of lesions was visually confirmed. As a result, as illustrated in FIG. 3A, it was confirmed that, when doxycycline was continuously administered to the Peli1 transgenic mice (rtTA-Peli1), lesions spontaneously occurred. In particular, hair gloss disappeared, hair loss progressed, and abnormal findings of the skin were observed.

Next, as a result of measuring the proportion of mice exhibiting the above-described phenotype, as illustrated in FIG. 3B, it was confirmed that, when Peli1 was overexpressed according to doxycycline administration, the above-described lesions were shown in all of the mice.

Next, control (rtTA) mice and experimental (rtTA-Peli1) mice were administered doxycycline for 24 weeks, and shown changes in phenotype were scored, and at this time, scoring was performed based on standards shown in Table 1. As a result, as illustrated in FIG. 3C, it was confirmed that the score continued to increase in accordance with the administration period of doxycycline.

TABLE 1

| Score | Phenotype |
|---|---|
| 0 | Normal |
| 1 | Slight erythema and keratinization |
| 2 | Increased area of erythema, keratinization, and the skin layer |
| | begins to become thick |
| 3 | Severe keratinization and erythema, and the skin layer is thickened |
| | by about 30% |
| 4 | Severe keratinization and erythema, and the skin layer is thickened |
| | by 60% or more than normal |
| 5 | Severe keratinization and erythema, and skin |
| | exfoliation are observed, and the skin layer is thickened |
| | by 90% or more than normal |

From the above results, it was seen that the above-described lesions were spontaneously induced by the overexpression of Peli1.

### Example 5. Verification of Psoriasis Induction of Transgenic Mice Inducibly Expressing Peli1

To confirm whether psoriasis was induced in the transgenic mice inducibly expressing Peli1, which were produced in Example 2, the following experiments were carried out.

5-1. Confirmation of Change in Layer of Epidermal Cells

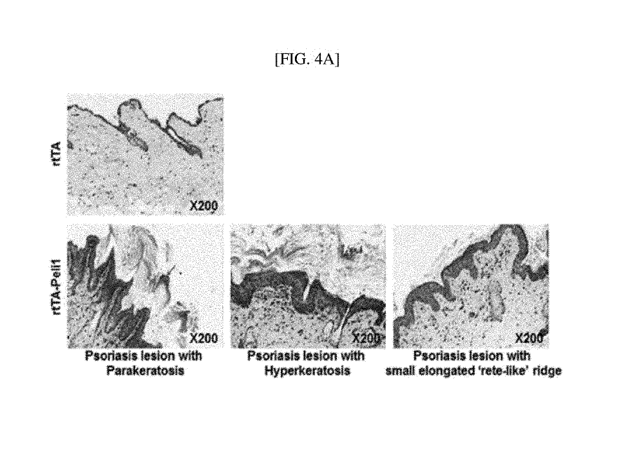

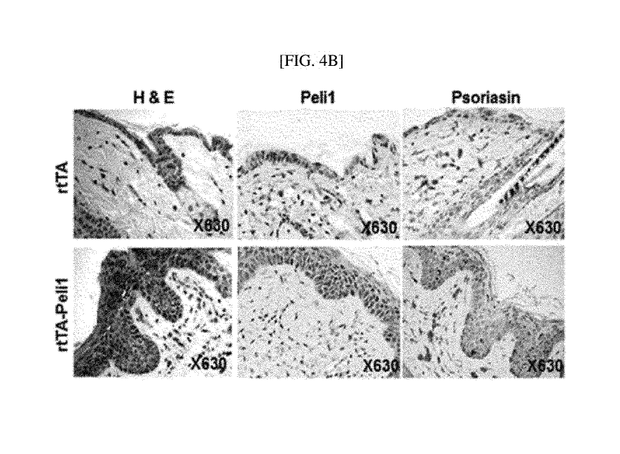

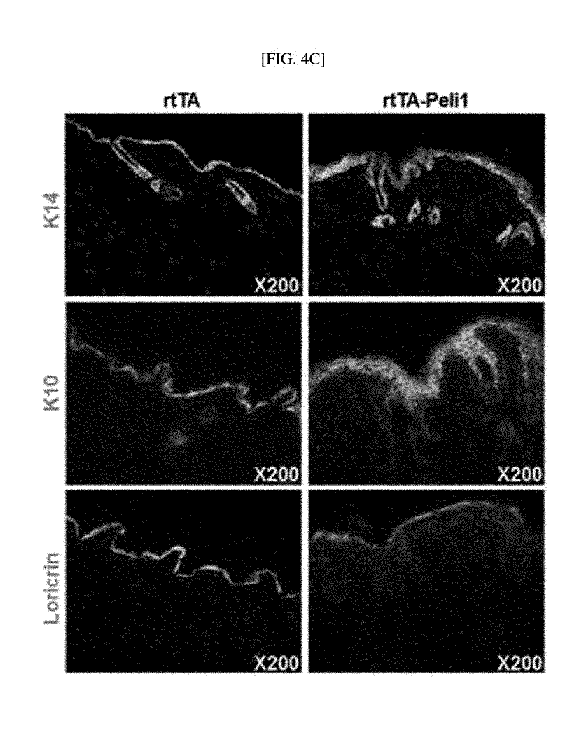

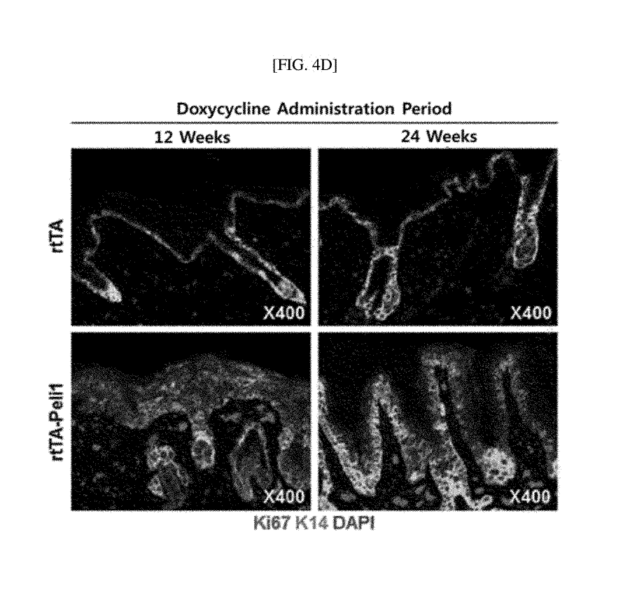

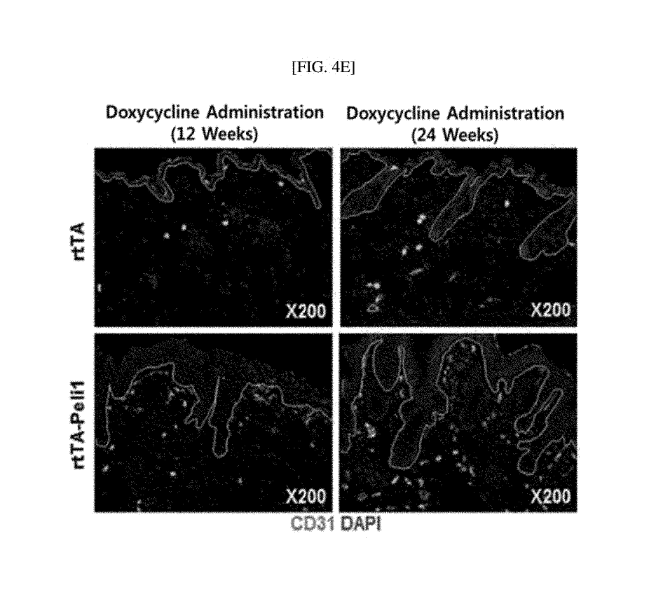

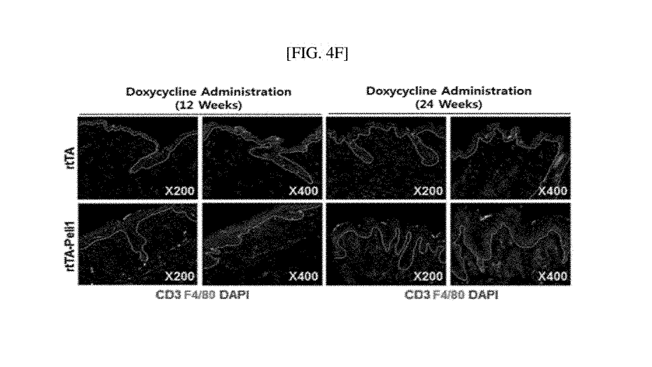

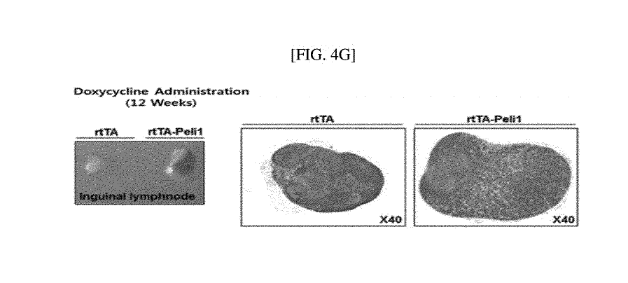

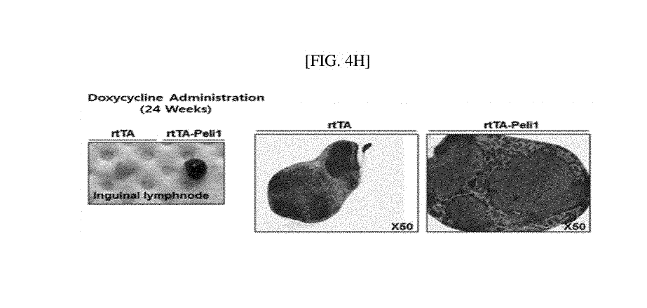

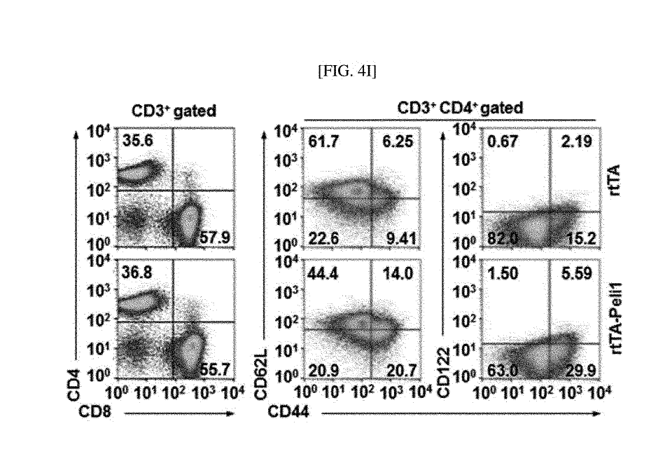

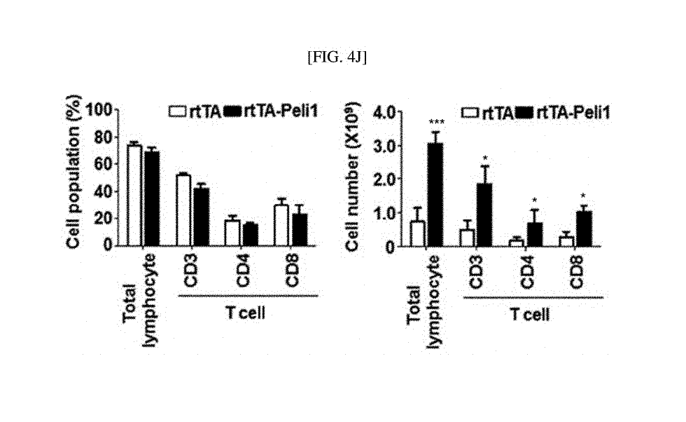

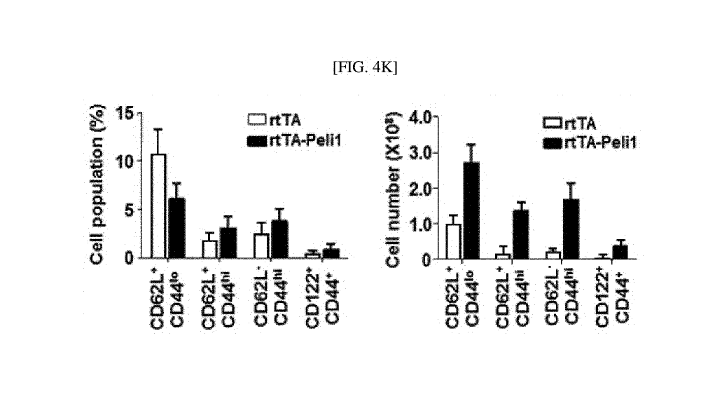

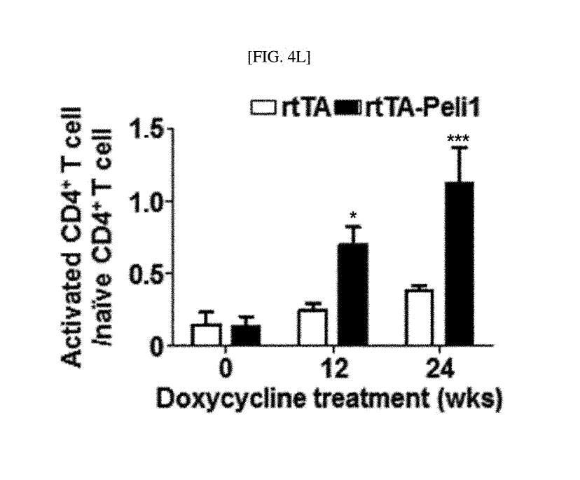

First, control (rtTA) mice and experimental (rtTA-Peli1) mice were administered drinking water containing doxycycline (2 mg/ml) for 24 weeks, and structures of the skin layers were identified through H&E staining as described in Example 1-5. As a result, as illustrated in FIG. 4A, while normal skin layers were formed in the control (rtTA) mice, abnormal lesions were observed in the experimental (rtTA-Peli1) mice. In particular, the layer of epidermal cells was significantly increased, and phenotypes commonly found in skin layer structures of patients with psoriasis, such as parakeratosis, hyperkeratosis, rete ridge, and microabscess, were observed. From the above results, it was confirmed that the phenotype of psoriasis was observed in the transgenic mice inducibly expressing Peli1. In addition, control (rtTA) mice and experimental (rtTA-Peli1) mice were administered drinking water containing doxycycline (2 mg/ml) for 24 weeks, and then immunohistologic staining was performed using Peli1 antibodies to confirm the overexpression of the Peli1 protein in the skin layer. As a result, as illustrated in FIG. 4B, it was confirmed that the Peli1 protein was overexpressed in the skin layers of the experimental (rtTA-Peli1) mice. In addition, as a result of performing immunohistologic staining using an antibody against Psoriasin, which is a psoriasis marker protein, the expression of Psoriasin was strongly induced in the experimental (rtTA-Peli1) mice administered doxycycline. Next, through the results of FIG. 4A, it was confirmed that, when doxycycline was continuously administered to the Peli1 transgenic mice, the layer of epidermal cells was abnormally thickened, in contrast to the control, and thus immunostaining was performed using antibodies against K14, K10, and Loricrin, which are markers of the basal layer, the spinous layer, and the granular layer, respectively, of the epidermal layer, according to the method described in Example 1-5 to examine the stained images. As a result, as illustrated in FIG. 4C, it was confirmed that the basal layer and spinous layer of the epidermal layer were significantly increased in the experimental (rtTA-Peli1) mice, as compared to those in the control (rtTA) mice.

In addition, to confirm the proliferative capacity of basal cells, the number of which was abnormally increased, in the Peli1 transgenic mice, the cells were stained with Ki67, which is a marker for confirming cell proliferation, according to the method described in Example 1-5, and the stained images were examined. As a result, as illustrated in FIG. 4D, it was confirmed that the number of basal cells being proliferated was increased in the experimental (rtTA-Peli1) mice, as compared to that in the control (rtTA) mice. From the above results, it was confirmed that the number of basal cells being proliferated was abnormally increased in the Peli1 transgenic mice (rtTA-Peli1), and, accordingly, the layer of epidermal cells was thickened.

5-2. Confirmation of Change in Angiogenesis of Epidermal Layer

In addition to abnormal thickening of the epidermal layer, abnormally and actively occurring angiogenesis in the dermal layer and the occurrence of angiectasis are the phenotypes found in patients with psoriasis. To confirm them, first, staining was performed using an antibody against CD31, which is a marker for capillary vessels, according to the method described in Example 1-5. As a result, as illustrated in FIG. 4E, it was confirmed that angiogenesis actively occurred and angiectasis was shown in the experimental (rtTA-Peli1) mice, as compared to the control (rtTA) mice.

5-3. Confirmation of Change in Activation of Immune Cells

Actively occurring infiltration of immune cells is one of the phenotypes found in patients with psoriasis. In addition, according to recent studies, it has been known that psoriasis is induced by abnormal activity of T cells, and thus the infiltration of T cells is observed in the skin layer.

To confirm this, first, staining was performed using an antibody against CD3, which is a marker for T cells, according to the method described in Example 1-5. As a result, as illustrated in FIG. 4F, it was confirmed that the infiltration of T cells into the skin layer was increased in the experimental (rtTA-Peli1) mice, as compared to that in the control (rtTA) mice.

Next, from the results of FIG. 4F, the induction of abnormal immune responses could be expected, and thus lymph nodes around skin lesions were identified. More particularly, inguinal lymph nodes, axillary lymph nodes, brachial lymph nodes, and cervical lymph nodes, which are present close to the outermost skin layer, were separated to examine structures thereof through the H&E staining described in Example 1-5. As a result, as illustrated in FIGS. 4G and 4H, it was confirmed that lymph nodes around lesions inflated in the experimental (rtTA-Peli1) mice, as compared to those in the control (rtTA) mice. In addition, it was confirmed that some lymph nodes turned black, from which it was seen that the formation of sinuses was abnormally increased due to an increase in the activity of immune cells.

Next, through the results of FIGS. 4G and 4H, it was confirmed that lymph nodes around skin lesions inflated, and thus the activation of immune cells in these lymph nodes was examined by the flow cytometry described in Example 1-4.

More particularly, immune cells were isolated from lymph nodes, and the activity of CD4⁺ T cells among T cells was examined using CD44, CD62L, and CD122 antibodies. As a result, as illustrated in FIG. 4I, it was confirmed that a reduced proportion of naïve T cells (CD62L⁺/CD44⁻) and an increased proportion of activated T cells (CD62L⁺/CD44^{hi-low}, CD62L⁻/CD44^{hi}, CD122⁺/CD44⁺) were shown in the experimental (rtTA-Peli1) mice, as compared to those in the control (rtTA) mice.

In addition, as a result of analyzing the results of FIG. 4I as a graph, as illustrated in FIG. 4J, a difference in the proportion of T cells constituting the lymph nodes was not observed, but as a result of comparing the numbers of cells, it was confirmed that not only an overall increase in the number of immune cells, but also a significant increase in the number of CD3⁺, CD4⁺, CD8⁺ T cells were shown in the Peli1 transgenic mice (rtTA-Peli1).

In addition, as a result of analyzing the proportion and number of the activated T cells using a graph, as illustrated in FIGS. 4K and 4L, it was confirmed that due to the overall increase in the number of immune cells in the lymph nodes of the Peli1 transgenic mice (rtTA-Peli1), the number of the naïve and activated T cells was increased, but as a result of examining the proportion, a ratio of the activated T cells to the naïve T cells was increased. From these results, it was seen that immune cells were abnormally activated in the Peli1 transgenic mice (rtTA-Peli1) according to the present disclosure.

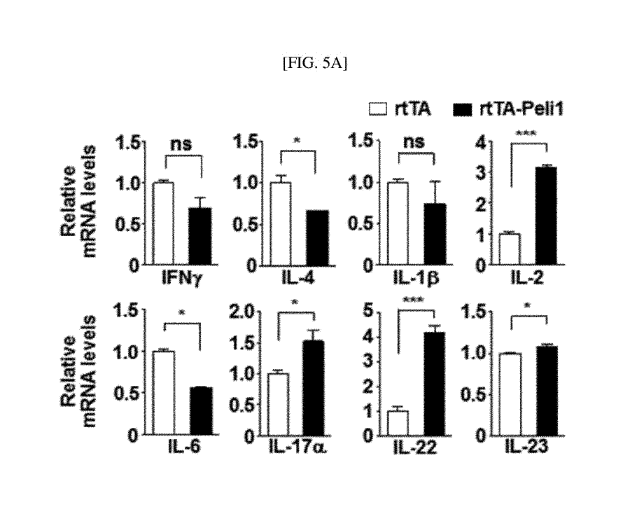

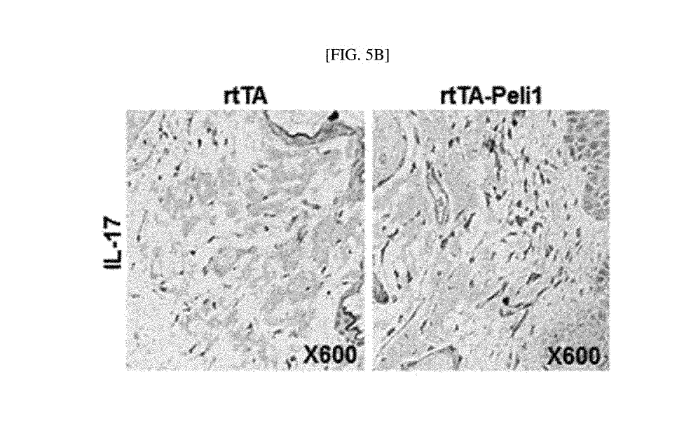

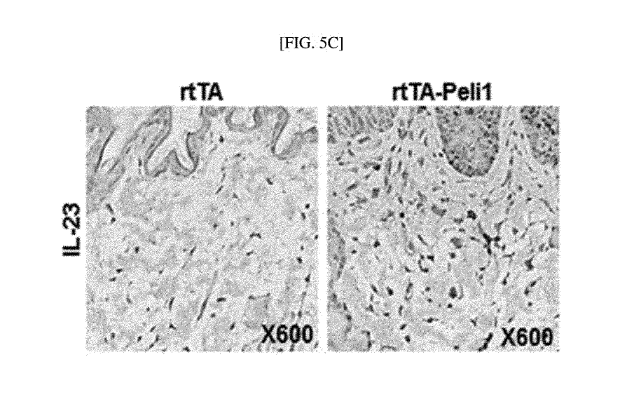

5-4. Confirmation of Change in Helper T Response of T Cells According to a previous report, it is known that abnormal helper T responses are induced in psoriasis animal models and patients. Thus, the levels of cytokines, such as IFNγ, IL-4, IL-22, and IL-23, were compared with each other by quantitative reverse transcription polymerase chain reaction using cDNA of CD3⁺ T cells of the lymph nodes. As a result, as illustrated in FIG. 5A, it was confirmed that the levels of IFNγ, which is a cytokine secreted in a type 1 helper T response, and IL-22 and IL-23, which are cytokines secreted in a type 17 helper T response, were significantly increased.

In addition, to confirm whether an abnormal helper T response was also induced in the skin layer, the immunostaining described in Example 1-5 was performed using antibodies against IL-17 and IL-23. As a result, as illustrated in FIGS. 5B and 5C, it was confirmed that the number of cells stained with the antibody against IL-17 or IL-23 was significantly increased in the Peli1 transgenic mice (rtTA-Peli1), as compared to that in the control (rtTA) mice.

These are very similar to the phenotypes shown in psoriasis, and from these results, it was confirmed that immune cells in the Peli1 transgenic mice (rtTA-Peli1) according to the present disclosure were abnormally activated and also underwent an abnormal helper T response.

5-5. Comparison in Similarity to Lesions Shown in Patients with Psoriasis

Based on the results of analyzing various lesions shown in the Peli1 transgenic mice (rtTA-Peli1) through Examples 5-1 to 5-4, similarities to the phenotypes shown in patients with psoriasis were compared with each other. As a result, as shown in Table 2 below, it was confirmed that the phenotypes shown in the Peli1 transgenic mice (rtTA-Peli1) according to the present disclosure were very similar to the phenotypes shown in patients with psoriasis.

TABLE 2

| | | | | | Vascular | Inflamatory | | | | |
|---|---|---|---|---|---|---|---|---|---|---|
| | | | | | changes | changes | | | Phenotype | |
| | Epidermal changes | | | | Dilation | | Intra- | | dependent | Features |
| | | Altered | | | of | Epidermal | epidermal | “Koebner” | on | unlike |
| | Thick- | differen- | Rete | Papillo- | Capillary | T-cell | micro- | Phenom- | Phenotype | human |
| Model | ening | tiation | ridges | matosis | loops | infiltrate | abscesses | enon | activation | psoriasis |
| Human | Y | Y | Y | N | Y | Y | Y | Y | Y | |
| psoriasis | | | | | | | | | | |
| Peli1 | Y | Y | Y | N | Y | Y | Y | ? | Y | Retarded |
| Inducible | | | | | | | | | | growth, |
| TG | | | | | | | | | | hair |
| | | | | | | | | | | abnormalities |

### Example 6. Verification of the Possibility of Peli1 as Novel Target for Psoriasis Treatment

Based on the results of Examples 4 and 5, the possibility of psoriasis induction due to overexpression of the Peli1 protein was confirmed, and thus the following experiments were carried out to confirm the possibility of the Peli1 protein to treat psoriasis by inhibiting the expression of the Peli1 protein.

6-1. Verification of Possibility of Psoriasis Treatment Via Doxycycline Administration

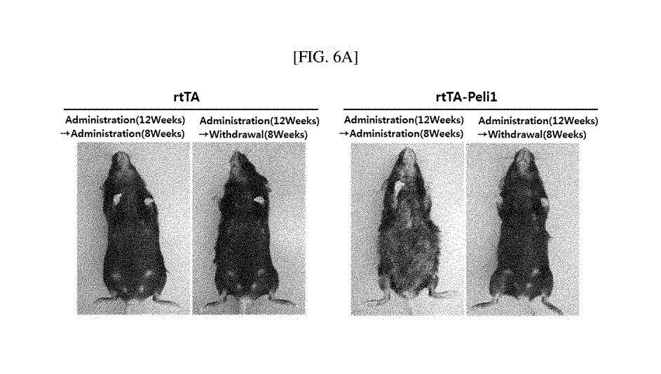

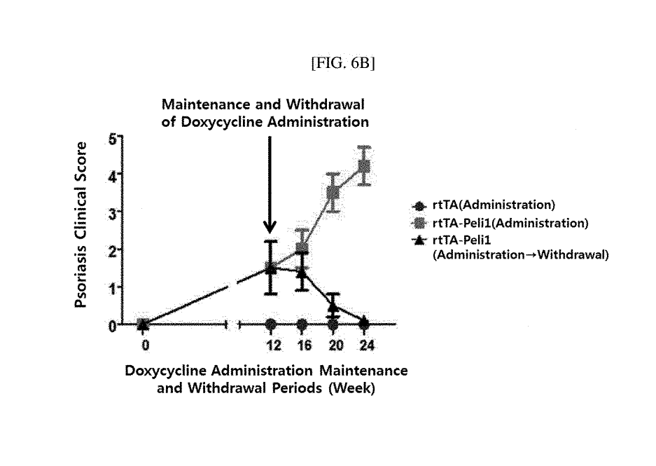

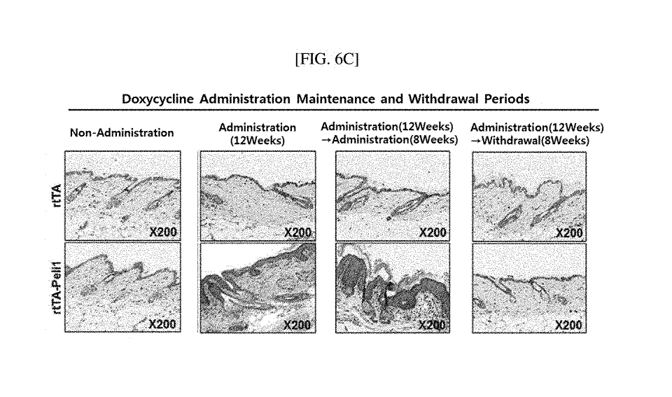

The phenotypes of psoriasis were analyzed when the phenotypes of psoriasis appeared via the administration of doxycycline to control mice (rtTA) and experimental mice (rtTA-Peli1) for a certain period of time and when the expression of Peli1 was reduced to a normal level according to withdrawal of doxycycline. As a result, as illustrated in FIG. 6A, it was confirmed that while the phenotypes of psoriasis appeared in the Peli1 transgenic mice (rtTA-Peli1) when doxycycline was continuously administered for 5 months, the phenotypes of psoriasis disappeared and phenotypes similar to those of the control mice (rtTA) was shown when doxycycline was administered for 3 months, followed by the withdrawal of doxycycline.

Next, the control (rtTA) mice and the experimental (rtTA-Peli1) mice were administered doxycycline for 12 weeks, and then changes in phenotypes shown while doxycycline was administered again and removed again were scored. As a result, as illustrated in FIG. 6B, it was confirmed that the score continued to increase and decrease according to doxycycline administration and withdrawal periods.

According to the results of FIGS. 6A and 6B, H&E staining as described in Example 1-5 was performed to confirm the structures of skin layers. As a result, as illustrated in FIG. 6C, the layer of epidermal cells was significantly increased in the Peli1 transgenic mice (rtTA-Peli1) according to doxycycline administration, and phenotypes commonly found in skin layer structures of patients with psoriasis, such as parakeratosis, hyperkeratosis, rete ridge, and microabscess, were observed. However, it was confirmed that the phenotypes of parakeratosis, hyperkeratosis, and rete ridge disappeared in the skin layer structures of the Peli1 transgenic mice (rtTA-Peli1) after doxycycline administration and the withdrawal of doxycycline, and were also very similar to those in skin layer structures of the control mice (rtTA).

From the above results, it was confirmed that an abnormally increased expression of Peli1 could induce the onset of psoriasis, and it was confirmed that phenotypes of the skin layer that were very similar to those of normal cases were shown when the expression of Peli1 was reduced again to a normal level, and thus the possibility of Peli1 as a novel target for psoriasis treatment was confirmed.

6-2. Confirmation of Lesion Levels According to Psoriasis Induction when Peli1 Expression was Inhibited

From the results of Example 6-1, it was confirmed that, when the abnormally increased expression of Peli1 was reduced to a normal level, the level of a psoriasis lesion was reduced to a normal level, and thus the following experiments were carried out to confirm lesion levels according to psoriasis induction when the expression of Peli1 was inhibited at a normal level.

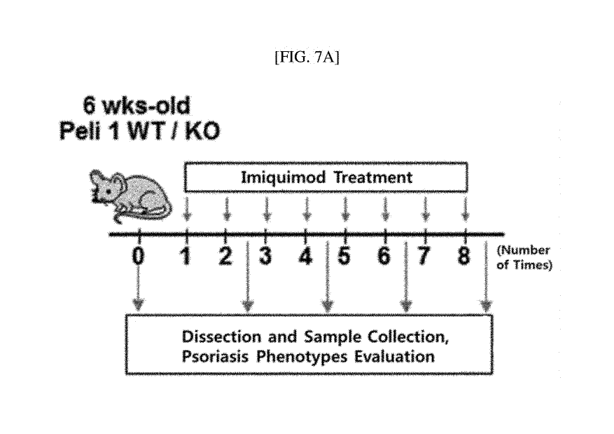

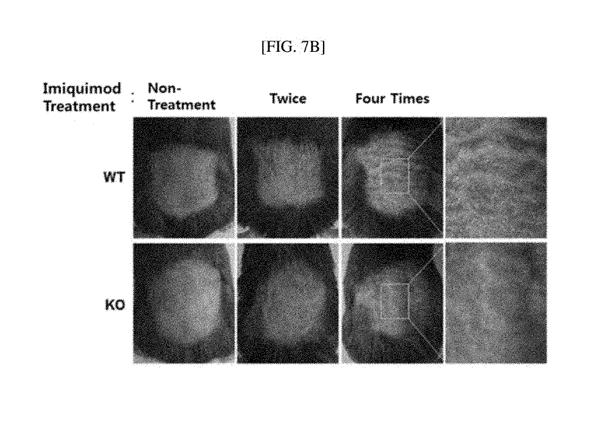

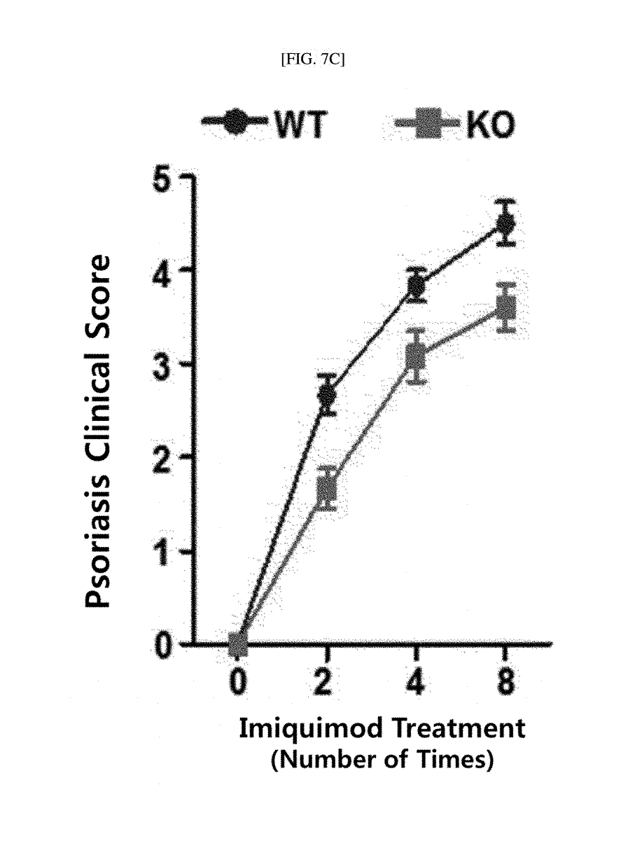

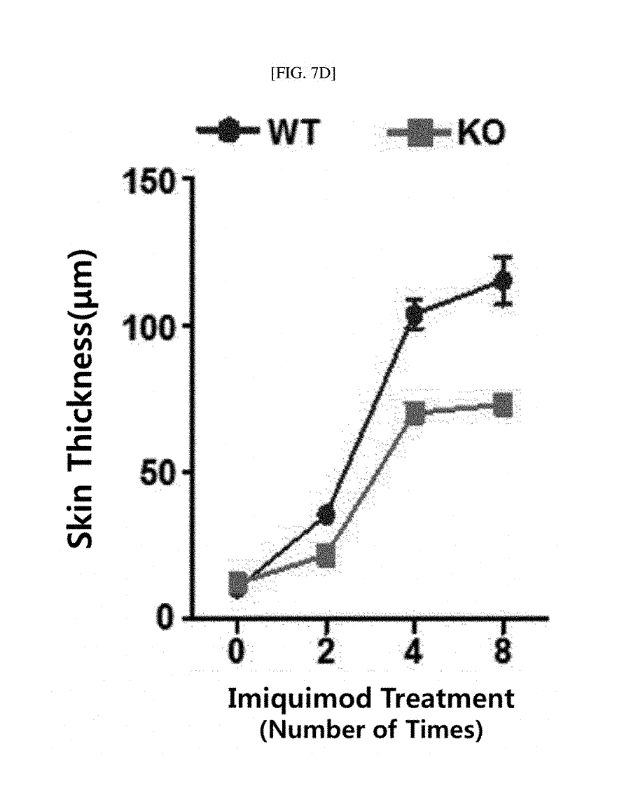

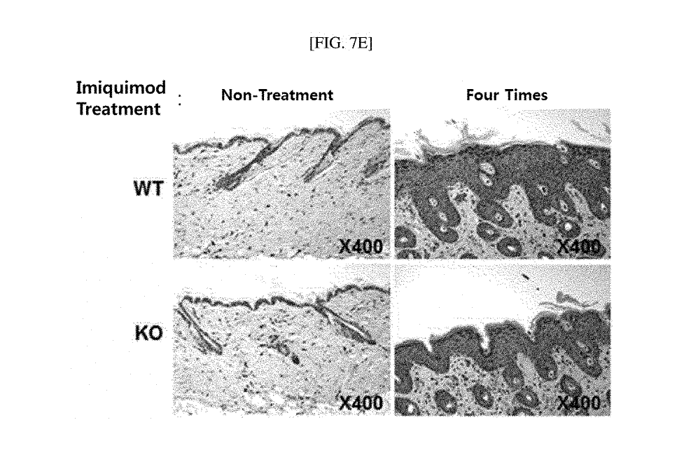

In particular, for an experiment, dorsal hair-removed mice were treated with 62.5 mg of imiquimod every day by using a psoriasis animal model using imiquimod (IMQ), which is an agonist of TLR7 or TLR9, in normal mice (WT) and Peli1 knockdown mice (Peli1 KO) (see FIG. 7A).

As a result, as illustrated in FIG. 7B, it was confirmed that the phenotypes of psoriasis, such as erythema and keratosis, were shown in the normal mice (WT) according to imiquimod treatment, but the Peli1 knockdown mice (Peli1 KO) exhibited erythema while exhibiting a lower level of keratosis than that in the normal mice (WT).

Next, the normal mice (WT) and the Peli1 knockdown mice (Peli1 KO) were treated with imiquimod, and then changes in psoriasis phenotypes were scored. As a result, as illustrated in FIG. 7C, it was confirmed that while the score was increased in the normal mice (WT) according to imiquimod treatment, the Peli1 knockdown mice (Peli1 KO) exhibited a lower score than that of the normal mice.

According to the results of FIGS. 7B and 7C, H&E staining as described in Example 1-5 was performed to confirm the structures of skin layers. As a result, as illustrated in FIGS. 7D and 7E, it was confirmed that while the thickness of the skin was increased in the normal mice (WT) according to imiquimod treatment, the thickness of the skin was less increased in the Peli1 knockdown mice (Peli1 KO) than in the normal mice. In addition, it was confirmed that the Peli1 knockdown mice (Peli1 KO) had a smaller rete ridge depth than that of the normal mice (WT), and hyperkeratosis was also alleviated in the Peli1 knockdown mice (Peli1 KO) more than in the normal mice (WT).

From the above results, it was confirmed that, as a result of comparing the phenotypes of psoriasis lesions of the normal mice (WT) and the Peli1 knockdown mice (Peli1 KO) by using an existing psoriasis animal model by imiquimod treatment, the levels of psoriasis lesions were reduced by imiquimod treatment when Peli1 expression was inhibited, as compared to the normal mice, and thus the possibility of Peli1 as a novel target for psoriasis treatment was confirmed again.

### Example 7. Verification of Use of Peli1 Transgenic Mice for Preclinical Psoriasis Animal Model

Based on the results of Examples 4 and 5, the possibility of psoriasis induction due to overexpression of the Peli1 protein according to the administration of doxycycline to Peli1 transgenic mice was confirmed, and thus verification of the Peli1 transgenic mice as a preclinical psoriasis animal model for screening for a psoriasis therapeutic agent and verification of the effect thereof was performed.

To this end, psoriasis is alleviated using an existing potent immunosuppressant, and mechanisms of cyclosporine (CsA) and methotrexate (MTX), which are systemic therapeutic agents used in patients with psoriasis, are as follows. Cyclosporine (CsA) inhibits the function of NFAT by inhibiting the activity of calcineurin that induce the activity of T cells, and thus reduces the secretion of a specific cytokine, thereby inhibiting the activity of T cells.

In addition, methotrexate (MTX) inhibits the hyperdivision of epithelial cells, the induction of apoptosis of activated T cells, the chemotaxis of neutrophils, and the secretion of specific cytokines.

According to this, it was examined whether the psoriasis phenotypes of the Peli1 transgenic mice were alleviated when cyclosporine (CsA) or methotrexate (MTX) was administered, and whether the Peli1 transgenic mice were applicable as a preclinical animal model.

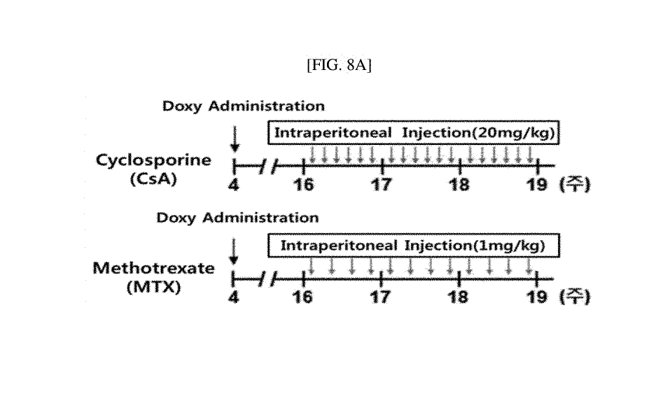

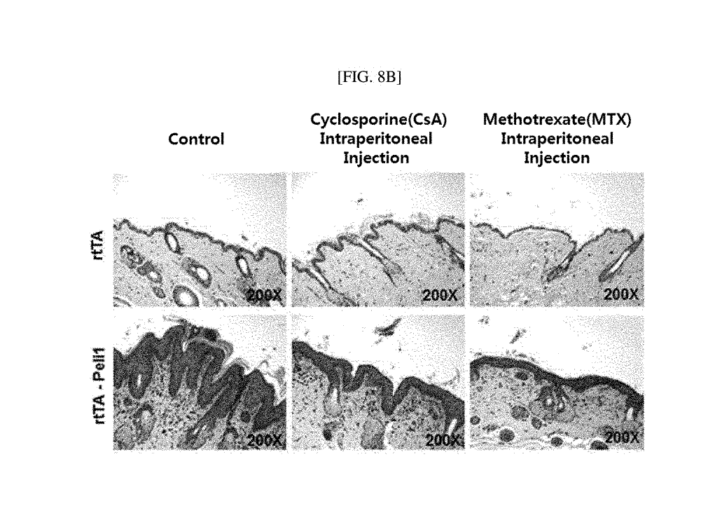

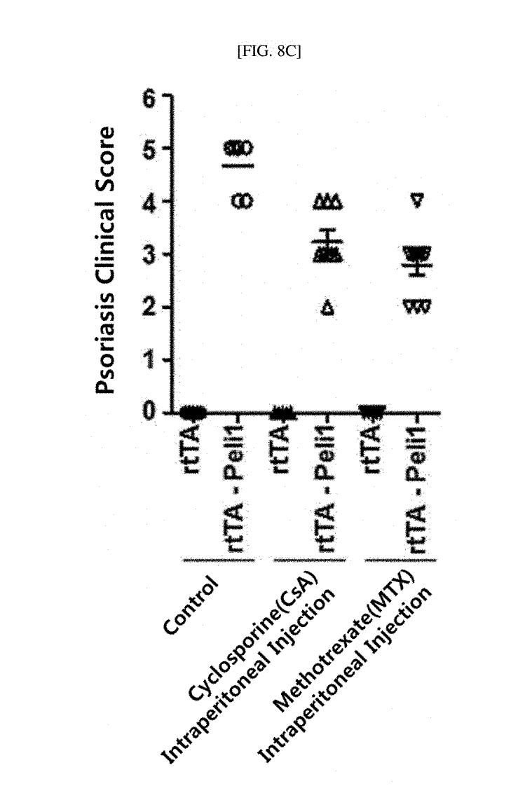

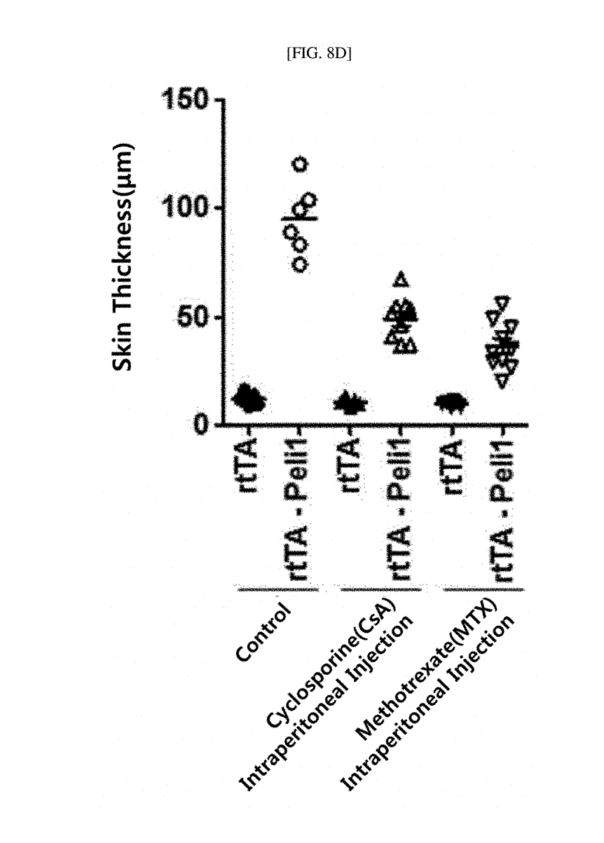

To this end, as illustrated in FIG. 8A, when the phenotypes of psoriasis appeared via the administration of doxycycline to control mice (rtTA) and experimental mice (rtTA-Peli1) for a certain period of time (12 weeks), cyclosporine (CsA) was administered 6 times a week and methotrexate (MTX) was intraperitoneally injected 4 times a week, and then the phenotypes of psoriasis were analyzed. To confirm the structures of skin layers, H&E staining as described in Example 1-5 was performed.

As a result, as illustrated in FIG. 8B, it was confirmed that most of the phenotypes of parakeratosis, hyperkeratosis, rete ridge, and microabscess shown in the Peli1 transgenic mice (rtTA-Peli1) disappeared after administration of cyclosporine (CsA) or methotrexate (MTX), which are immunosuppressants. However, it was confirmed that an increase in the thickness of the skin according to the proliferation of epidermal cells was still observed even after administration of cyclosporine (CsA) or methotrexate (MTX), which are immunosuppressants.

Next, when the phenotypes of psoriasis appeared via the administration of doxycycline to the control mice (rtTA) and the experimental mice (rtTA-Peli1) for a certain period of time (12 weeks), cyclosporine (CsA) or methotrexate (MTX), which are existing immunosuppressants used for treatment in patients, was intraperitoneally injected for a certain period of time, and then the psoriasis phenotypes were scored.

As a result, as illustrated in FIG. 8C, it was confirmed that the psoriasis phenotype score was reduced when cyclosporine (CsA) or methotrexate (MTX), which are immunosuppressants, was intraperitoneally injected for a certain period of time.

From the results of FIG. 8B, it was confirmed that, after cyclosporine (CsA) or methotrexate (MTX), which are immunosuppressants, was intraperitoneally injected for a certain period of time, most psoriasis phenotypes disappeared, but an increase in skin thickness according to the proliferation of epidermal cells was still observed. As such, a change in skin thickness was observed.

As a result, as illustrated in FIG. 8D, it was confirmed that, after cyclosporine (CsA) or methotrexate (MTX), which are immunosuppressants, was intraperitoneally injected into the Peli1 transgenic mice wherein the phenotypes of psoriasis appeared according to doxycycline administration, for a certain period of time, about half of the thickness of the skin was reduced.

From the above results, it was confirmed that the effect of a psoriasis therapeutic agent was verified using the Peli1 transgenic mice exhibiting the phenotypes of psoriasis according to doxycycline administration, and thus a decrease in the psoriasis phenotypes was confirmed. Thus, the possibility of the Peli1 transgenic mice as a preclinical animal model for screening for a novel psoriasis therapeutic agent and verification of the effect thereof was verified.

### Example 8. Preparation of Peli1 Protein Expression Inhibitor

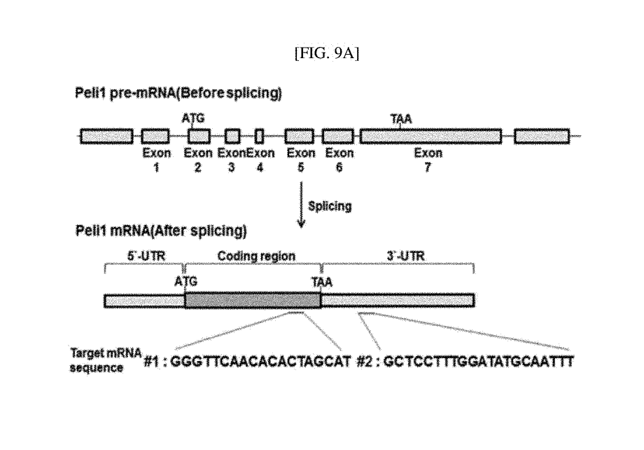

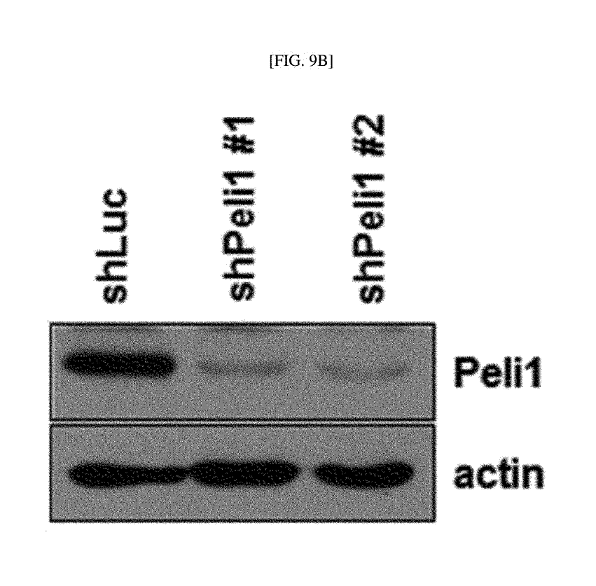

Based on the results of Example 6, the possibility of psoriasis treatment by the inhibition of Peli1 protein expression was verified, and thus to inhibit protein synthesis in Peli1 mRNA, target mRNA sequences were identified (see FIG. 9A), and, based on this, a shRNA and a siRNA were prepared as follows.

(SEQ ID NO: 3)
Target mRNA sequence #1: GGGTTCAACACACTAGCAT
(SEQ ID NO: 4)
Target mRNA sequence #2: GCTCCTTTGGATATGCAATTT

Next, as a result of transfecting cells with the prepared shRNA and examining the expression of the Peli1 protein through western blotting as described in Example 1-3, as illustrated in FIG. 9B, it was confirmed that in the case of transfection of cells with the shRNA targeting Peli1 mRNA, the expression of the Peli1 protein was effectively inhibited.

### Example 9. Preparation of Peptide Inhibiting Binding Activity of Peli1

9-1. Preparation of Peptide Inhibiting Binding Activity of Peli1

Peli1, which is an E3 ubiquitin-conjugating enzyme, exhibits conjugating enzymatic activity through the RING-like domain and binds to a target protein through the FHA domain Thus, to inhibit the binding between Peli1 and the target protein, a peptide derived from the FHA domain of the Peli1 protein was prepared.

TABLE 3

| | FHA domain-derived peptide sequence |
|---|---|
| 95-114 | SNTDMFQIGRSTESPIDFVV |
| 101-116 | QIGRSTESPIDFVVTD |
| 109-124 | PIDFVVTDTVPGSQSN |
| 117-132 | TVPGSQSNSDTQSVQS |
| 125-140 | SDTQSVQSTISRFACR |
| 133-149 | TISRFACRIICERNPP |
| 177-196 | DGQMDGLTTNGVLVMHPRNG |

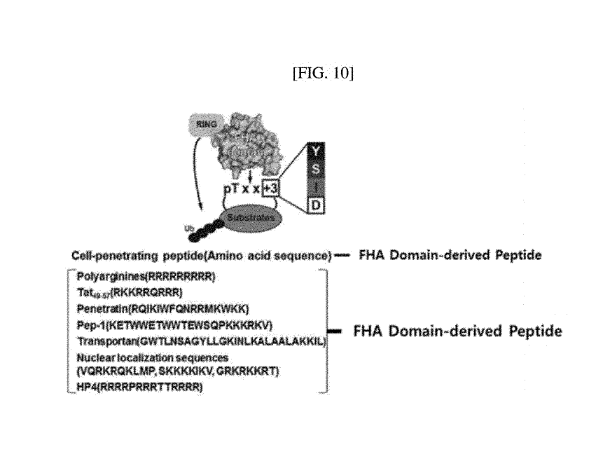

In addition, to facilitate the intracellular penetration of the prepared peptide, cell-penetrating peptides were linked thereto, and the used cell-penetrating peptides are as follows (see FIG. 10):

| | |
|---|---|
| i) | Polyarginines (RRRRRRRRR) |
| ii) | Tat₄₉₋₅₇ (RKKRRQRRR) |
| iii) | Penetratin (RQIKIWFQNRRMKWKK) |
| iv) | Pep-1 (KETWWETWWTEWSQPKKKRKV) |
| v) | Transportan (GWTLNSAGYLLGKINLKALAALAKKIL) |
| vi) | Nuclear localization sequences (VQRKRQKLMP, |
| | SKKKKIKV, GRKRKKRT) |
| vii) | HP4 (RRRRPRRRTTRRRR) |

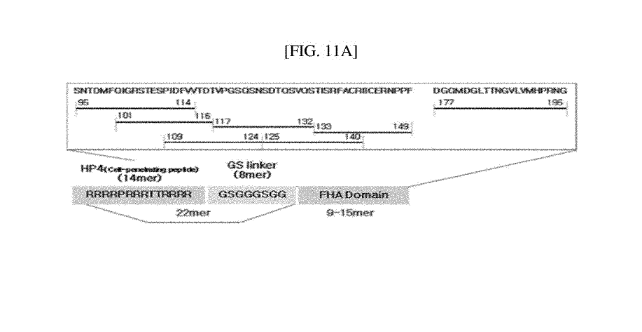

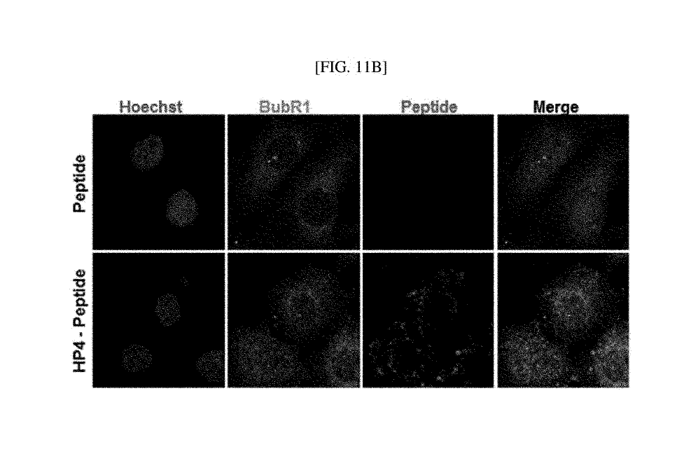

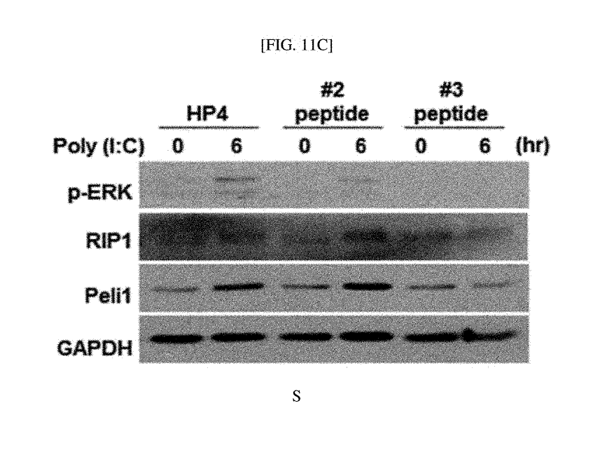

Based on FIG. 10, to produce a peptide inhibiting the binding activity of Peli1, as depicted in FIG. 11A, the peptide inhibiting the binding activity of Peli1 was produced using the FHA domain, HP4, which is a cell-penetrating peptide, and a GS linker sequence connecting the two peptides to each other.

9-2. Verification of Cell Penetrability of Peptide Inhibiting Binding Activity of Peli1

To confirm the intracellular penetrability of the peptide inhibiting the binding activity of Peli1 produced according to Example 9-1, the peptide inhibiting the binding activity of Peli1 was treated with a cell culture, and then the intracellular penetrability was examined through fluorescence staining.

As a result, as illustrated in FIG. 11B, it was confirmed that the intracellular penetrability was significantly enhanced when HP4, which is a cell-penetrating peptide, was linked. From this, it was confirmed that the peptide inhibiting the binding activity of Peli1, to which the cell-penetrating peptide was linked, infiltrated into cells.

9-3. Confirmation of Function of Peptide Inhibiting Binding Activity of Peli1

Next, to verify the function of the peptide inhibiting the binding activity of Peli1 that infiltrated into cells, confirmed in Example 9-2, the following experiment was performed.

In particular, macrophages derived from bone marrow cells were treated with Poly(I:C), which is mediated by Peli1 and a TLR3 signaling agonist, and then the activation of TLR3 signaling was compared.

As a result, as illustrated in FIG. 11C, it was specifically confirmed that, upon treatment with Poly(I:C), which is a TLR3 signaling agonist, after the infiltration of the peptide inhibiting the binding activity of Peli1 into cells, the activity of TLR3 signaling was inhibited.

The foregoing description of the present disclosure is provided for illustrative purposes only, and it will be understood by those of ordinary skill in the art to which the present disclosure pertains that the present disclosure may be easily modified in other particular forms without changing the technical spirit or essential characteristics of the present disclosure. Thus, the above-described embodiments should be construed as being provided for illustrative purposes only and not for purposes of limitation.

### INDUSTRIAL APPLICABILITY

A transgenic animal model according to the present disclosure is anticipated to be usefully used in clinical studies such as screening for a candidate drug for the treatment of psoriasis, and the like. In addition, a peptide derived from the Peli1 FHA domain targeting the FHA binding motif that inhibits normal substrate binding between a substrate protein and the Peli1 protein is anticipated to be usefully used in the development of new drugs for psoriasis-associated diseases.

1. A psoriasis-induced transgenic animal model overexpressing a Pellino homolog 1 (Peli1) gene.

2. The psoriasis-induced transgenic animal model of claim 1, wherein the Peli1 gene is overexpressed according to administration of doxycycline.

3. The psoriasis-induced transgenic animal model of claim 1, wherein the Peli1 gene has a base sequence of SEQ ID NO: 1.

4. A method of producing a psoriasis-induced animal model, the method comprising the following processes:
(a) transfecting an embryo of a host animal with an expression vector comprising the Pellino homolog 1 (Peli1) gene;
(b) implanting the transfected embryo into the uterus of a surrogate mother and allowing the surrogate mother to give birth to the embryo as a first generation progeny animal; and
(c) crossing the first generation progeny animal with a reverse tetracycline transactivator (rtTA) animal model to obtain a second generation progeny animal.

5. A method of screening for a psoriasis therapeutic agent, the method comprising the following processes:
a) treating the animal model of claim 1 with a candidate material; and
b) identifying a prognosis while rearing the animal model treated with the candidate material.

6-10. (canceled)

11. A peptide having any one amino acid sequence selected from the group consisting of SEQ ID NOS: 5 to 11.

12. The peptide of claim 11, wherein the peptide inhibits binding between the Peli1 protein and a substrate protein.

13. A method of treating psoriasis, comprising:
administering to a subject in need thereof an effective amount of the peptide of claim 11.

14. A peptide inhibiting the binding activity of Peli1, characterized in that a cell-penetrating peptide is linked to the peptide of claim 11.

15. The peptide of claim 14, wherein the peptide inhibiting the binding activity of Peli1 is linked via a GS linker.

16. The peptide of claim 14, wherein the cell-penetrating peptide is any one selected from the group consisting of polyarginines, Tat₄₉₋₅₇, penetratin, Pep-1, transportan, nuclear localization sequence (NLS), and HP4.

17. A method of treating psoriasis, comprising:
administering to a subject in need thereof an effective amount of the peptide inhibiting the binding activity of Peli1 of claim 14.

18-24. (canceled)


SEQUENCE LISTING (17 sequences)

SEQ ID NO: 1 - DNA, 1257 bp, Homo sapiens
  Pellino homolog 1
atgttttctcctgatcaagaaaatcatccatctaaagcaccagtaaaatatggtgaactcattgtcttagggtataatgggtctctcccaaatggcgatagaggaaggaggaaaagtaggtttgctttgtttaaaagacctaaggcaaatggggtgaagcccagcactgtgcatattgcttgtactcctcaggctgcaaaggcaataagcaacaaagaccagcatagcatatcatatactttatctcgggcccagactgtggtggttgaatatactcatgacagcaacaccgatatgtttcagattggccggtcgactgaaagccccattgattttgtagtaactgacacggttcctggaagtcaaagtaattctgatacacagtcagtacaaagcactatatcaagatttgcctgcagaatcatatgtgaacggaatcctccctttacagcacggatttatgctgcaggatttgactcatcaaaaaacatctttcttggggagaaggctgccaaatggaagacatcagatggacagatggatggcttgaccactaatggtgttcttgtgatgcatccacgcaatgggttcacagaagactccaagcctggaatatggagagaaatatcggtgtgtggaaatgtatttagcctacgtgaaaccagatcggctcagcagagaggaaaaatggtggaaattgaaaccaatcagttacaagatggctcgttaattgacctctgtggtgcaacattgttatggcgtactgcagaaggcctttcccacactcctaccgtgaagcatttagaagctttaagacaggaaatcaatgcagcacgacctcagtgccctgtagggttcaacacactagcatttcctagtatgaagaggaaagacgttgtagatgaaaaacaaccatgggtatatctaaactgcggccatgtacatggctatcataactggggaaacaaagaagaacgtgatggaaaagatcgtgaatgtcctatgtgtaggtctgttggtccctatgttcctctgtggcttggatgtgaagctggattttatgtggacgccggccctccaacccatgcgtttagcccgtgtgggcatgtgtgttcagaaaagacaactgcctattggtcccagatcccacttcctcatggtactcatacttttcatgcagcctgtcccttttgtgcacatcagttggctggtgaacaaggctacatcagacttatttttcaaggacctctagactaa

SEQ ID NO: 2 - protein, 418 aa, Homo sapiens
  Pellino homolog 1
MFSPDQENHPSKAPVKYGELIVLGYNGSLPNGDRGRRKSRFALFKRPKANGVKPSTVHIACTPQAAKAISNKDQHSISYTLSRAQTVVVEYTHDSNTDMFQIGRSTESPIDFVVTDTVPGSQSNSDTQSVQSTISRFACRIICERNPPFTARIYAAGFDSSKNIFLGEKAAKWKTSDGQMDGLTTNGVLVMHPRNGFTEDSKPGIWREISVCGNVFSLRETRSAQQRGKMVEIETNQLQDGSLIDLCGATLLWRTAEGLSHTPTVKHLEALRQEINAARPQCPVGFNTLAFPSMKRKDVVDEKQPWVYLNCGHVHGYHNWGNKEERDGKDRECPMCRSVGPYVPLWLGCEAGFYVDAGPPTHAFSPCGHVCSEKTTAYWSQIPLPHGTHTFHAACPFCAHQLAGEQGYIRLIFQGPLD

SEQ ID NO: 3 - DNA, 19 bp, Artificial Sequence
  shRNA of Peli1
gggttcaacacactagcat

SEQ ID NO: 4 - DNA, 21 bp, Artificial Sequence
  siRNA of Peli1
gctcctttggatatgcaattt

SEQ ID NO: 5 - protein, 20 aa, Artificial Sequence
  FHA Binding Peptide 1
SNTDMFQIGRSTESPIDFVV

SEQ ID NO: 6 - protein, 16 aa, Artificial Sequence
  FHA Binding Peptide 2
QIGRSTESPIDFVVTD

SEQ ID NO: 7 - protein, 16 aa, Artificial Sequence
  FHA Binding Peptide 3
PIDFVVTDTVPGSQSN

SEQ ID NO: 8 - protein, 16 aa, Artificial Sequence
  FHA Binding Peptide 4
TVPGSQSNSDTQSVQS

SEQ ID NO: 9 - protein, 16 aa, Artificial Sequence
  FHA Binding Peptide 5
SDTQSVQSTISRFACR

SEQ ID NO: 10 - protein, 16 aa, Artificial Sequence
  FHA Binding Peptide 6
TISRFACRIICERNPP

SEQ ID NO: 11 - protein, 20 aa, Artificial Sequence
  FHA Binding Peptide 7
DGQMDGLTTNGVLVMHPRNG

SEQ ID NO: 12 - DNA, 18 bp, Artificial Sequence
  TetO & Peli 1 forward primer
aagtgaaagtcgagctcg

SEQ ID NO: 13 - DNA, 22 bp, Artificial Sequence
  TetO & Peli 1 reverse primer
tgatatcgtgtgctggtctttg

SEQ ID NO: 14 - DNA, 21 bp, Artificial Sequence
  R26 & rtTA forward primer
aaagtcgctctgagttgttat

SEQ ID NO: 15 - DNA, 21 bp, Artificial Sequence
  R26 & rtTA reverse primer
ggagcgggagaaatggatatg

SEQ ID NO: 16 - DNA, 21 bp, Artificial Sequence
  R26 & rtTA forward primer 2
aaagtcgctctgagttgttat

SEQ ID NO: 17 - DNA, 21 bp, Artificial Sequence
  R26 & rtTA reverse primer 2
gcgaagagtttgtcctcaacc